SEQUENCE LISTING (39 sequences)

SEQ ID NO: 1 - protein, 845 aa, hepatitis B virus
MPLSYQHFRKLLLLDDGTEAGPLEEELPRLADADLNRRVAEDLNLGNLNVSIPWTHKVGNFTGLYSSTVPIFNPEWQTPSFPKIHLQEDIINRCQQFVGPLTVNEKRRLKLIMPARFYPTHTKYLPLDKGIKPYYPDQVVNHYFKTRHYLHTLWKAGILYKRETTRSASFCGSPYSWEQELQHGRLVIETSQRHGDESFCSQPSGILSRSSVGPCIRSQLKQSRLGLQPHQGPLASSQPGRSGSIRARVHPSTRRCFGVEPSGSGHIDHSVNNSSSCLHQSAVRKAAYSHLSTSKRQSSSGHAVEFHCLPPSSAGSQSQGSVFSCWWLQFRNSKPCSEYCLSHLVNLREDWGPCDEYGEHHIRIPRTPARVTGGVFLVDKNPHNTAESRLVVDFSQFSRGITRVSWPKFAVPNLQSLTNLLSSNLSWLSLDVSAAFYHIPLHPAAMPHLLVGSSGLSRYVARLSSNSRNNNNQYGTMQNLHDSCSRQLYVSLMLLYKTFGWKLHLYSHPIVLGFRKIPMGVGLSPFLLAQFTSAICSVVRRAFPHCLAFSYMDDVVLGAKSVQHRESLYTAVTNFLLSLGIHLNPNKTKRWGYSLNFMGYIIGSWGTLPQDHIVQKIKHCFRKLPVNRPIDWKVCQRIVGLLGFAAPFTQCGYPALMPLYACIQAKQAFTFSPTYKAFLSKQYMNLYPVARQRPGLCQVFADATPTGWGLAIGHQRMRGTFVAPLPIHTAELLAACFARSRSGAKLIGTDNSVVLSRKYTSFPWLLGCTANWILRGTSFVYVPSALNPADDPSRGRLGLSRPLLRLPFQPTTGRTSLYAVSPSVPSHLPVRVHFASPLHVAWRPP

SEQ ID NO: 2 - protein, 843 aa, hepatitis B virus
MPLSYQHFRKLLLLDEEAGPLEEELPRLADEGLNRRVAEDLNLGNLNVSIPWTHKVGNFTGLYSSTVPCFNTNWQTPSFPDIHLQEDIVDRCKQFVGPLTVNEKRRLKLIMPARFYPNVTKYLPLDKGIKPYYPEHVVNHYFQTKHYLHTLWKAGILYKRESTRSASFCGSPYSWEQDLQHGRLVFQTSKRHEDKSFCPQSPGILPRSSVGSCIQSQLRKSRLGPQPAQGQLAGRQQGGSGSIRARVHPSPWGTVGVEPSGSGHTHNCASSSSSCLHQSAVRRAAYSLISTAKGHSSSGHAVELHHFPPNSSRSQSQGPVLSCWWLQFRNSEPCSEYCLCHIVNIIEDWGPCTEHGEHRIRTPRTPARVTGGVFLVDKNPHNTKESRLVVDFSQFSRGNTRVSWPKFAVPNLQSLTNLLSSNLSWLSLDVSAAFYHLPLHPAAMPHLLVGSSGLSRYVARLSSNSRIINNQHRTMQNLHNSCSRKLYVSLMLLYKTYGRKLHLYSHPIILGFRKIPMGVGLSPFLLAQFTSAICSVVRRAFPHCLAFSYMDDVVLGAKSVQHLESLYAAVTNFLLSLGIQLNPHKTKRWGYSLNFMGYVIGGWGTLPQEHIVQKIKMCFRKLPVNRPIDWKVCQRIVGLLGFAAPFTQCGYPALMPLYACIQAKQAFTFSPTYKAFLSKQYLNLYPVARQRSGLCQVFADATPTGWGLAIGHQRMRGTFVSPLPIHTAELLAACFARSRSGAKLIGTDNSVVLSRKYTSFPWLLGCAANWILRGTSFVYVPSALNPVDDPSRGRLGLYRPLLRLLYRPTTGRTSLYADSPSVPSHLPDRVHFASPLHVAWRPP

SEQ ID NO: 3 - protein, 843 aa, hepatitis B virus
MPLSYQHFRKLLLLDDEAGPLEEELPRLADEGLNRRVAEDLNLGNLNVSIPWTHKVGNFTGLYSSTVPVFNPEWQTPSFPNIHLQEDIINRCQQYVGPLTINEKRRLKLIMPARFYPNLTKYLPLDKGIKPYYPEHAVNHYFKTRHYLHTLWKAGILYKRETTRSASFCGSPYSWEQELQHGRLVFQTSTRHGDESFCSQSSGIFSRSPVGPSIRSQLKQSRLGLQPQQGSLARGKSGRSGSIRARVHPTTRWSFGVEPSGSGHIDNSASSASSCLHQSAVRKTAYSHLSTSKRQSSSGHAVELHHIPPSSARSQSEGPIFSCWWLQFRNSKPCSDYCLTHIVNLLEDWGPCTEHGEHNIRIPRTPARVTGGVFLVDKNPHNTTESRLVVDFSQFSRGSTHVSWPKFAVPNLQSLTNLLSSNLSWLSLDVSAAFYHIPLHPAAMPHLLVGSSGLPRYVARLSSTSRNINYQHGTMQDLHDSCSRNLYVSLLLLYKTFGRKLHLYSHPIILGFRKIPMGVGLSPFLLAQFTSAICSVVRRAFPHCLAFSYMDDVVLGAKSVQHLESLFTSITNFLLSLGIHLNPNKTKRWGYSLNFMGYVIGSWGTLPQEHIVLKLKQCFRKLPVNRPIDWKVCQRIVGLLGFAAPFTQCGYPALMPLYACIQAKQAFTFSPTYKAFLCQQYLHLYPVARQRSGLCQVFADATPTGWGLAIGHRRMRGTFVAPLPIHTAELLAACFARSRSGATLIGTDNSVVLSRKYTSFPWLLGCAANWILRGTSFVYVPSALNPADDPSRGRLGLYRPLLHLPFRPTTGRTSLYAVSPSVPSHLPDRVHFASPLHVAWKPP

SEQ ID NO: 4 - protein, 832 aa, hepatitis B virus
MPLSYQHFRRLLLLDDEAGPLEEELPRLADEGLNRRVAEDLNLGNLNVSIPWTHKVGNFTGLYSSTVPVFNPHWKTPSFPNIHLHQDIIKKCEQFVGPLTVNEKRRLQLIMPARFYPNVTKYLPLDKGIKPYYPEHLVNHYFQTRHYLHTLWKAGILYKRETTHSASFCGSPYSWEQELQHGAESFHQQSSGILSRPPVGSSLQSKHRKSRLGLQSQQGHLARRQQGRSRSIRAGIHPTARRPFGVEPSGSGHTTNLASKSASCLYQSPVRKAAYPSVSTFEEHSSSGHAVELHNLPPNSARSQSERPVFPCWWLQFRNSKPCSDYCLSHIVNLLEDWGPCAEYGEHHIRIPRTPARVTGGVFLVDKNPHNTAESRLVVDFSQFSRGNYRVSWPKFAVPNLQSLTNLLSSNLSWLSLDVSAAFYHLPLRPAAMPHLLVGSSGLSRYLARLSSNSRIFNHQHGTMQNLHDSCSRNLYVSLLLLYQTFGRKLHLYSHPIILGFRKIPMGVGLSPFLLAQFTSAICSVVRRAFPHCLAFSYMDDVVLGAKSVQHLESLFTAVTNFLLSLGIHLNPNKTKRWGYSLHFMGYVIGCYGSLPQDHIIQKIKECFRKLPVNRPIDWKVCQRIVGLLGFAAPFTQCGYPALMPLYACIQSKQAFTFSPTYKAFLCKQYLNLYPVARQRPGLCQVFADATPTGWGLVMGHQRMRGTFLAPLPIHTAELLAACFARSRSGANILGTDNSVVLSRKYTSFPWLLGCAANWILRGTSFVYVPSALNPADDPSRGRLGLSRPLLRLPFRPTTGRTSLYADSPSVPFHLPDRVHFASPLHVAWRPP

SEQ ID NO: 5 - protein, 842 aa, hepatitis B virus
MPLSYQHFRRILLLDEEAGPLEEELPRLADEDLNRRVAEDLNLQLPNVSIPWTHKVGNFTGLYSSTIPVFNPNWKTPSFPDIHLHQDIINKCEQFVGPLTVNEKRRLNLVMPARFFPISTKYLPLEKGIKPYYPDNVVNHYFQTRHYLHTLWKAGHLYKRETTRSASFCGSPYSWEQELHHGAFLDGPSRMGEEYFHHQSSGIFSRPPVGSSIQSKHQKSRLGPQSQQRPLDGSQQGRSGSLRAGVHSPTRRPFGVEPSGSRHAKNIASRPASCLHQSAVRKAAYPNHSTFERHSSSGHAVELHNISSSSAGSQSKRPVFSCWWLQFRNSEPCSDYCLTHLVNLLEDWGPCTEHGKHHIRIPRTPARVTGGVFLVDKNPHNTAESRLVVDFSQFSRGSSRVSWPKFAVPNLQSLTNLLSSNLSWLSLDVSAAFYHLPLHPAAMPHLLVGSSGLSRYVARLSSNSRIINHQHGTLPNLHDSCSRNLYVSLMLLFKTFGRKLHLYSHPIIMGFRKIPMGVGLSPFLLAQFTSAICSVVRRAFPHCLAFSYMDDVVLGAKSVRHLESLYTSVTNFLLSLGIHLNPNKTKRWGYSLNFMGYVIGSWGSLPQEHIIQKIKHCFGKLPVNRPIDWKVCQGIVGLLGFAAPFTQCGYPALMPLYTCIQSKQAFTFSPTYKAFLCKQYLNLYPVARQRPGLCQVFADATPTGWGLAIGIQRMRGTFVAPLPIHTAELLAACFARSRSGAKLIGTDNSVVLSRKYTSFPWLLGCAANWILRGTSFVYVPSALNPADDPSRGRLGIFRPLLRLRFRPTTGRTSLYAVSPSVPSHLPDRVHFASPLHVAWRPP

SEQ ID NO: 6 - protein, 843 aa, hepatitis B virus
MPLSYPHFRKLLLLDDEAGPLEEELPRLADEGLNRRVAEDLNLQLPNVSIPWTHKVGNFTGLYSSTVPTFNPDWLTPSFPDIHLHQDLIHKCEQFVGPLTKNELRRLKLVMPSRFFPKVTKYFPMEKGIKPYYPDNVVNHYFKTRHYLHTLWKAGILYKRESTRSASFCGSPYSWEQELQHGSTSINDSKGHGTESLCTQSSGILSRPSAGSSIQGKFQQSRLGLQQKQGQLANGKQGRSGRIRSWVHTPTRWPVGVESTGTGCAHNIASRSASCFHQSAVREKTNPSLSTSKRHSSTGHAVELHSVPPGSVRSEGKGSVFSCWWLQFRDTEPCSDYCLSHIINLLEDWGPCYEHGQHHIRTPRTPARVTGGVFLVDKNPHNTTESRLVVDFSQFSRGNTRVSWPKFAVPNLQSLTNLLSSNLSWLSLDVSAAFYHLPLHPAAMPHLLVGSSGLSRYVARLSSTSRIHDHQHGTMQNLHNSCSRNLYVSLLLLFQTLGRKLHLYSHPIILGFRKIPMGVGLSPFLLAQFTSAICSVVRRAFPHCLAFSYMDDLVLGAKSVQHLESLYTAVTNFLLSVGIHLNTSKTKRWGYTLNFMGYVIGSWGSLPQDHIVQKIKDCFRKLPVNRPIDWKVCQRIVGLLGFAAPFTQCGYPALMPLYACITAKQAFVFSPTYKAFLCQQYMNLYPVARQRPGLCQVFADATPTGWGLAIGHQRMRGTFVAPLPIHTAELLAACFARSRSGAKLIGTDNSVVLSRKYTSFPWLLGCAANWILRGTSFVYVPSALNPADDPSRGRLGLYRPLLRLPFQPTTGRTSLYAASPSVPSHLPDRVHFASPLHVAWRPP

SEQ ID NO: 7 - protein, 840 aa, hepatitis B virus
MPLSYQHFRKLLLLDDGTEAGPLEEELPRLADADLNRRVAEDLNLGNLNVSIPWTHKVGNFTGLYSSTVPIFNPEWQTPSFPKIHLQEDIINRCQQFVGPLTVNEKRRLKLIMPARFYPTHTKYLPLDKGIKPYYPDQVVNHYFKTRHYLHTLWKAGILYKRETTRSASFCGSPYSWEQELQHGRLVIETSQRHGDESFCSQPSGILSRSSVGPCIRSQLKQSRLGLQPHQGPLASSQPGRSGSIRARVHPSTRRCFGVEPSGSGHIDHSVNNSSSCLHQSAVRKAAYSHLSTSKRQSSSGHAVEFHCLPPSSAGSQSQGSVFSCWWLQFRNSKPCSEYCLSHLVNLREDWGPCDEYGEHHIRIPRTPARVTGGVFLVDKNPHNTAESRLVVDFSQFSRGITRVSWPKFAVPNLQSLTNLLSSNLSWLSLDVSAAFYHIPLHPAAMPHLLVGSSGLSRYVARLSSNSRNNNNQYGTMQNLHDSCSRQLYVSLMLLYKTFGWKLHLYSHPIVLGFRKIPMGVGLSPFLLAQFTSAICSVVRRAFPHCLAFSYMDDVVLGAKSVQHRESLYTAVTNFLLSLGIHLNPNKTKRWGYSLNFMGYIIGSWGTLPQDHIVQKIKHCFRKLPVNRPIDWKVCQRIVGLLGFAAPFTQCGYPALMPLYACIQAKQAFTFSPTYKAFLSKQYMNLYPVARQRPGLCQVFADATPTGWGLAIGHQRMRGTFVAPLPIHTAELLAACFARSRSGAKLIGTDNSVVLSRKYTSFPWLLGCTANWILRGTSFVYVPSALNPADDPSRGRLGLSRPLLRLPFQPTTGRTSLYAVSPSVPSHLPVRVHFASPLHV

SEQ ID NO: 8 - protein, 843 aa, hepatitis B virus
MPLSYQHFRRLLLLDNEAGPLEEELPRLADEDLNLRVAEDLNLQLPNVSIPWTHKVGNFTGLYSSTIPVFNPDWLTPSFPDIHLHQDLIQKCEQFVGPLTTNERRRLKLIMPARFYPKVTKYFPLDKGIKPYYPENVVNHYFKTRHYLHTLWKAGILYKRESTHSASFCGSPYSWEQELQHGSTSLNGEKGHGTESFCAQSSGILSRPPVGSTIQSKFQQSRLGLQHKQGQLANGKQGRSGRLRSRVHTPTRWPSGVEPSGTGHSDNLATRSTSCFHQSEVRKKANPSLSTSKGHTSTGRAVELNTVPPSTVGSESKGAVFSCWWLQFRNTEPCSDYCLSRIVNLLEDWGPCYEHGEHHIRTPRTPSRVTGGVFLVDKNPHNTKESRLVVDFSQFSRGTTRVSWPKFAVPNLQSLTNLLSSNLSWLSLDVSAAFYHLPLHPAAMPHLLVGSSGLSRYVARVSSTSRIYNHQHGTLQNLHHSCSRNLYVSLLLLYQTFGRKLHLYSHPIILGFRKIPMGVGLSPFLLAQFTSAICSVVRRAFPHCLAFSYMDDLVLGAKSVQHLESLYTAVTNFLLSVGIHLNTAKTKWWGYTLHFMGYRIGSWGTLPQEHIVHKIKDCFRKLPVNRPIDWKVCQRIVGLLGFAAPFTQCGYPALMPLYACITAKQAFVFSPTYKAFLCKQYMNLYPVARQRPGLCQVFADATPTGWGLAIGHQRMRGTFVAPLPIHTAELLAACFARSRSGADIIGTDNSVVLSRKYTSFPWLLGCAANWILRGTSFVYVPSALNPADDPSRGRLGLYRPLLRLAFRPTTGRTSLYADSPPVPSHLPARVHFASPLHVAWRPP

SEQ ID NO: 9 - DNA, 11 bp, hepatitis B virus
ttcacctctgc

SEQ ID NO: 10 - DNA, 11 bp, hepatitis B virus
ttcacctctgc

SEQ ID NO: 11 - DNA, 61 bp, hepatitis B virus (genetype A epsilon)
tgtacatgtcccactgttcaagcctccaagctgtgccttgggtggctttggggcatggaca

SEQ ID NO: 12 - DNA, 61 bp, hepatitis B virus (genetypes B-F epsilon)
tgttcatgtcctactgttcaagcctccaagctgtgccttgggtggctttggggcatggaca

SEQ ID NO: 13 - DNA, 61 bp, hepatitis B virus (genetype G epsilon)
tgttcatgtcctactgttcaagcctccaagctgtgccttgggtggctttagggcatggata

SEQ ID NO: 14 - DNA, 61 bp, hepatitis B virus (genetype H epsilon)
tgttcatgtcccactgttcaagcctccaagctgtgccttgggtggctttggggcatggaca

SEQ ID NO: 15 - DNA, 1615 bp, Artificial
  synthetic
  misc_feature (1)..(6): BglII site
  misc_feature (7)..(747): CMV promoter
  misc_feature (748)..(793): synthetic
  misc_feature (794)..(892): HBV Genotype C 32-130
  misc_feature (893)..(898): EcoRI site
  misc_feature (899)..(1188): reporter sequence
  misc_feature (1189)..(1212): cloning site
  misc_feature (1213)..(1561): HBV Genotype C 3013-3215/1-146
  misc_feature (1562)..(1609): poly A signal
  misc_feature (1610)..(1615): EcoRI site
agatctcaatattggccattagccatattattcattggttatatagcataaatcaatattggctattggccattgcatacgttgtatctatatcataatatgtacatttatattggctcatgtccaatatgaccgccatgttggcattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtccgccccctattgacgtcaatgacggtaaatggcccgcctggcattatgcccagtacatgaccttacgggactttcctacttggcagtacatctacgtattagtcatcgctattaccatggtgatgcggttttggcagtacaccaatgggcgtggatagcggtttgactcacggggatttccaagtctccaccccattgacgtcaatgggagtttgttttggcaccaaaatcaacgggactttccaaaatgtcgtaataaccccgccccgttgacgcaaatgggcggtaggcgtgtacggtgggaggtctatataagcagagctcgtttagtgaaccgtcagatcactagaagctttattgcggtagtttatcacagttaaattgctaacggcaactttttcacctctgcctaatcatctcatgttcatgtcctactgttcaagcctccaagctgtgccttgggtggctttggggcatggacattgacccgaattcgcccttctcccagatggtgaacaggtcggtttccaggtaagtatcaaggttacaagacaggtttaaggagaccaatagaaactgggcttgtcgagacagagaagactcttgcgtttctgataggcacctattggtcttactgacatccactttgcctttctctccacaggttggtgatgccgaacttgccttccaggcccacgatgtcgatgttggggtcgaactcggtgctcttggcctgcaccagggcgctgaacaccagggtgaacaccaccttgatgtccaagggcgaattcacgcgtgtcgacctcgagcacttcgcttcacctctgcacgtcgcatggaaaccaccgtgaacacccaccaggtcttgcccaaggtcttacataagaggactcttggactctcagcaatgtcaacaaccgaccttgaggcatacttcaaagactgtttgtttaaagactgggaggagttgggggaggagattaggttaaaggtctttgtactaggaggctgtaggcataaattggtctgttcaccagcaccatgcaactttttcacctctgcctaatcatctcatgttcatgtcctactgttcaagcctccaagctgtgccttgggtggctttggggcatggacattgacccgtataaagaatttggaaataaaatatctttattttcattacatctgtgtgttggttttttgtgtggatcc

SEQ ID NO: 16 - protein, 185 aa, hepatitis B virus
MDIDPYKEFGATVELLSFLPSDFFPSVRDLLDTASALYREALESPEHCSPHHTALRQAILCWGELMTLATWVGNNLEDPASRDLVVNYVNTNMGLKIRQLLWFHISCLTFGRETVLEYLVSFGVWIRTPPAYRPPNAPILSTLPETTVVRRRDRGRSPRRRTPSPRRRRSQSPRRRRSQSRESQC

SEQ ID NO: 17 - protein, 183 aa, hepatitis B virus
MDIDPYKEFGASVELLSFLPSDFFPSIRDLLDTASALYREALESPEHCSPHHTALRQAILCWGELMNLATWVGSNLEDPASRELVVSYVNVNMGLKIRQLLWFHISCLTFGRETVLEYLVSFGVWIRTPPAYRPPNAPILSTLPETTVVRRRGRSPRRRTPSPRRRRSQSPRRRRSQSRESQC

SEQ ID NO: 18 - protein, 183 aa, hepatitis B virus; identical to SEQ ID NO: 17

SEQ ID NO: 19 - protein, 183 aa, hepatitis B virus
MDIDPYKEFGATVELLSFLPSDFFPSVRDLLDTASALYREALESPEHCSPHHTALRQAILCWGELMTLATWVGGNLEDPISRDLVVSYVNTNMGLKFRQLLWFHISCLTFGRETVIEYLVSFGVWIRTPPAYRPPNAPILSTLPETTVVRRRGRSPRRRTPSPRRRRSQSPRRRRSQSRESQC

SEQ ID NO: 20 - protein, 183 aa, hepatitis B virus
MDIDPYKEFGATVELLSFLPSDFFPSVRDLLDTASALYRDALESPEHCSPHHTALRQAILCWGELMTLATWVGVNLEDPASRDLVVSYVNTNMGLKFRQLLWFHISCLTFGRETVIEYLVSFGVWIRTPPAYRPPNAPILSTLPENTVVRRRGRSPRRRTPSPRRRRSQSPRRRRSQSPASQC

SEQ ID NO: 21 - protein, 183 aa, hepatitis B virus
MDIDPYKEFGASVELLSFLPSDFFPSVRDLLDTASALYRDALESPEHCTPNHTALRQAILCWGELMTLASWVGNNLEDPAARDLVVNYVNTHMGLKIRQLLWFHISCLTFGRETVLEYLVSFGVWIRTPPAYRPPNAPILSTLPETTVVRRRGRSPRRRTPSPRRRRSQSPRRRRSQSPASQC

SEQ ID NO: 22 - protein, 195 aa, hepatitis B virus
MDRTTLPYGLFGLDIDPYKEFGATVELLSFLPSDFFPSVRDLLDTASALYRESLESSDHCSPHHTALRQAILCWGELMTLATWVGNNLEDPASRDLVVNYVNTNMGLKIRQLLWFHISCLTFGRETVLEYLVSFGVWIRTPPAYRPPNAPILSTLPETTVVRRRGRSPRRRTPSPRRRRSQSPRRRRSASPASQC

SEQ ID NO: 23 - protein, 183 aa, hepatitis B virus
MDIDPYKEFGASAELLSFLPSDFFPSVRDLLDTASALYREALESPEHCTPNHTALRQAILCWGELMTLASWVGNNLQDPAARDLVVNYVNTNMGLKIRQLLWFHISCLTFGRETVLEYLVSFGVWIRTPPAYRPPNAPILSTLPETTVVRQRGRAPRRRTPSPRRRRSQSPRRRRSQSPASQC

SEQ ID NO: 24 - DNA, 552 bp, hepatitis B virus
atggacattgacccgtataaagaatttggagcatctgtggagttactctcttttttgccttctgacttctttccttctattcgagatctcctcgacaccgcctctgctctgtatcgggaggccttagagtctccggaacattgttcacctcaccatacagcactcaggcaagctattctgtgttggggtgagttaatgaatctggccacctgggtgggaagtaatttggaagacccagcatccagggaattagtagtcagctatgtcaatgttaatatgggcctaaaaatcagacaactattgtggtttcacatttcctgtcttacttttggaagagaaactgttcttgagtatttggtgtcttttggagtgtggattcgcactcctcccgcttacagaccaccaaatgcccctatcttatcaacacttccggaaactactgttgttagacgacgaggcaggtcccctagaagaagaactccctcgcctcgcagacgaaggtctcaatcgccgcgtcgcagaagatctcaatctcgggaatctcaatgttag

SEQ ID NO: 25 - DNA, 552 bp, Artificial
  synthetic
atggacatcgacccctacaaagaattcggcgccagcgtggaactgctgagcttcctgcccagcgacttcttcccctccatccgggacctgctggatacagccagcgccctgtatagagaggccctggaaagccccgagcactgcagccctcatcacaccgctctgagacaggccatcctgtgctggggcgagctgatgaatctggccacctgggtgggaagcaacctggaagatcccgccagccgggaactggtggtgtcctacgtgaacgtgaacatgggcctgaagatccggcagctgctgtggttccacatctcctgcctgaccttcggccgggaaaccgtgctggaatacctggtgtccttcggcgtgtggatcagaaccccccctgcctacagaccccccaacgcccctatcctgagcaccctgcctgagacaacagtcgtgcggcggagaggcagaagccccagaagaagaacccccagccccagacgcagaagaagccagtcccctcggcggagaagatcccagagcagggaaagccagtgctga

SEQ ID NO: 26 - DNA, 2532 bp, hepatitis B virus
atgcccctatcttatcaacacttccggaaactactgttgttagacgacgaggcaggtcccctagaagaagaactccctcgcctcgcagacgaaggtctcaatcgccgcgtcgcagaagatctcaatctcgggaatctcaatgttagtatcccttggactcacaaggtgggaaactttactgggctttattcttctactgtacctgtctttaatcctgagtggcaaactccctcctttcctaacattcatttacaggaggacattattaatagatgtcaacaatatgtgggccctcttacaattaatgaaaaaaggagattaaaattaattatgcctgctaggttttatcctaaccttaccaaatatttgcccttggataaaggcattaaaccttattatcctgaacatgcagttaatcattacttcaaaactaggcattatttacatactctgtggaaggctggcattctatataagagagaaactacacgcagcgcttcattttgtgggtcaccatattcttgggaacaagagctacagcatgggaggttggtcttccaaacctcgacaaggcatggggacgaatctttctgttcccaatcctctgggattttttcccgatcaccagttggacccagcattcggagccaactcaaacaatccagattgggacttcaaccccaacaaggatcactggccagaggcaaatcaggtaggagcgggagcattcgggccagggttcaccccaccacacggtggtcttttggggtggagccctcaggctcagggcatattgacaacagtgccagcagcgcctcctcctgcctccaccaatcggcagtcaggaagacagcctactcccatctctccacctctaagagacagtcatcctcaggccatgcagtggaactccaccacattccaccaagctctgctagatcccagagtgaggggcctatattttcctgctggtggctccagttccggaacagtaaaccctgttccgactactgcctcacccatatcgtcaaccttctcgaggactggggaccctgcaccgaacatggagaacacaacatcaggattcctaggacccctgctcgtgttacaggcggggtttttcttgttgacaagaatcctcacaataccacagagtctagactcgtggtggacttctctcaattttctagggggagcacccacgtgtcctggccaaaattcgcagtccccaacctccaatcactcaccaacctcttgtcctccaatttgtcctggctatcgttggatgtgtctgcggcgttttatcatattcctcttcatcctgctgctatgcctcatcttcttgttggttcttctggactaccaaggtatgttgcccgtttgtcctctacttccaggaacatcaactaccagcacgggaccatgcaagacctgcacgattcctgctcaaggaacctctatgtttccctcttgttgctgtacaaaaccttcggacggaaactgcacttgtattcccatcccatcatcctgggctttcgcaaaattcctatgggagtgggcctcagtccgtttctcctggctcagtttactagtgccatttgttcagtggttcgcagggctttcccccactgtttggctttcagttatatggatgatgtggtattgggggccaagtctgtacaacatcttgagtccctttttacctctattaccaattttcttttatctttgggtatacatttaaaccctaataaaaccaaacgttggggttactcccttaacttcatgggatatgtaattggaagttggggtactttaccacaggaacatattgtactaaaactcaagcaatgttttcgaaaactgcctgtaaatagacctattgattggaaagtatgtcaaagaattgtgggtcttttgggctttgctgccccttttacacaatgtggctatcctgccttgatgcctttatatgcatgtatacaagcgaagcaggctttcactttctcgccaacttacaaggcctttctgtgtcaacaatatctgcacctttaccccgttgcccggcaacggtcaggtctctgccaagtgtttgctgacgcaacccccactggttggggcttggccataggccatcggcgcatgcgtggaacctttgtggctcctctgccgatccatactgcggaactcctagcagcttgttttgctcgcagccggtctggagcgacacttatcgggactgacaactctgttgtcctctctcggaaatacacctccttcccatggctgctagggtgtgctgccaactggatcctgcgcgggacgtcctttgtctacgtcccgtcggcgctgaatcccgcggacgacccgtctcggggccgtttgggcctctaccgtccccttcttcatctgccgttccggccgaccacggggcgcacctctctttacgcggtctccccgtctgtgccttctcatctgccggaccgtgtgcacttcgcttcacctctgcacgtcgcatggaaaccaccgtga

SEQ ID NO: 27 - DNA, 2532 bp, Artificial
  synthetic
atgcctctgagctaccagcacttcagaaaactgctgctgctggacgacgaggccggacccctggaagaggaactgcctagactggccgacgagggcctgaacagaagagtggccgaggacctgaacctgggcaacctgaacgtgtccatcccctggacccacaaagtgggcaacttcaccggcctgtacagcagcaccgtgcccgtgttcaaccccgagtggcagacccccagcttccccaacatccatctgcaggaagatatcatcaaccgctgccagcagtacgtgggccccctgaccatcaacgagaagcggcggctgaagctgatcatgcccgccagattctaccccaacctgaccaagtacctgcccctggacaagggcatcaagccctactaccctgagcacgccgtgaaccactacttcaagacccggcactacctgcacaccctgtggaaggccggcatcctgtacaagagagagacaaccagaagcgccagcttctgcggcagcccctacagctgggagcaggaactgcagcacggccggctggtgtttcagaccagcacaagacacggcgacgagtctttctgcagccagagcagcggcatcttcagcagatccccagtgggccccagcatcagaagccagctgaagcagagcagactgggcctgcagcctcagcagggctctctggctagaggcaagagcggcagaagcggcagcatcagggccagagtgcaccctaccaccagatggtccttcggcgtggaacctagcggcagcggccacatcgataacagcgcctctagcgccagcagctgcctgcatcagtctgccgtgcggaaaaccgcctacagccacctgagcacctccaagagacagagcagctctggccacgccgtggaactgcaccacatccctccaagcagcgccagatctcagagcgagggccctatcttctcctgctggtggctgcagtttcggaacagcaagccctgcagcgactactgcctgacccacatcgtgaacctgctggaagattggggcccctgcacagagcacggcgagcacaacatccggatccccagaacccccgccagagtgaccggcggagtgttcctggtggacaagaacccccacaacaccaccgagagcaggctggtggtggacttcagccagttcagcagaggcagcacccacgtgtcctggcccaagttcgccgtgcccaatctgcagagcctgaccaacctgctgagcagcaacctgagctggctgagcctggatgtgtccgccgccttctatcacatccccctgcatcctgccgccatgccccatctgctcgtgggaagttctggcctgcccagatatgtggcccggctgtccagcacctcccggaacatcaattaccagcacggcaccatgcaggacctgcacgacagctgcagccggaatctgtacgtgtccctcctgctgctgtataagaccttcggccggaagctgcacctgtactcccaccccatcatcctgggcttcagaaagatccctatgggcgtgggcctgagcccattcctgctggcccagtttaccagcgccatctgtagcgtcgtgcggagagccttccctcactgcctggccttcagctacatggacgacgtggtgctgggcgccaagagcgtgcagcacctggaaagcctgtttacctccatcaccaactttctgctgtccctgggcatccacctgaaccccaacaagaccaagagatggggctacagcctgaacttcatgggctacgtgatcggcagctggggcaccctgcctcaggaacacatcgtgctgaaactgaagcagtgcttccgcaagctgcccgtgaaccggcccatcgactggaaagtgtgccagcggatcgtgggactgctgggctttgccgcccctttcacccagtgtggctaccccgctctgatgcccctgtacgcctgtatccaggccaagcaggccttcaccttcagccctacctacaaggccttcctgtgtcagcagtacctgcatctgtaccctgtggccagacagcggagcggactgtgtcaggtgttcgccgatgccacacctacaggctggggactggccatcggccacagaagaatgcggggcacctttgtggcccccctgcctattcacacagccgaactgctggccgcctgcttcgccagaagtagaagcggagccaccctgatcggcaccgacaactctgtggtgctgagccggaagtacaccagctttccctggctgctgggatgcgccgccaactggatcctgagaggcaccagcttcgtgtacgtgcccagcgccctgaaccctgccgacgatccttctagaggacggctgggcctgtaccggccactgctgcatctgcctttcagacccaccaccggccggaccagtctgtatgccgtgtctcctagcgtgccctcccatctgcccgacagagtgcatttcgccagccctctgcacgtggcctggaagcctccatga

SEQ ID NO: 28 - protein, 154 aa, hepatitis B virus
MAARLYCQLDPSRDVLCLRPVGAESRGRPLSGPLGTLSSPSPSAVPADHGAHLSLRGLPVCAFSSAGPCALRFTSARCMETTVNAHQILPKVLHKRTLGLPAMSTTDLEAYFKDCVFKDWEELGEEIRLKVFVLGGCRHKLVCAPAPCNFFTSA

SEQ ID NO: 29 - protein, 154 aa, hepatitis B virus
MAARLCCQLDPARDVLCLRPVGAESRGRPLPGPLGALPPASPPVVPTDHGAHLSLRGLPVCAFSSAGPCALRFTSARRMETTVNAHRNLPKVLHKRTLGLSAMSTTDLEAYFKDCVFTEWEELGEEIRLKVFVLGGCRHKLVCSPAPCNFFTSA

SEQ ID NO: 30 - protein, 154 aa, hepatitis B virus
MAARVCCQLDPARDVLCLRPVGAESRGRPVSGPFGPLPSPSSSAVPADHGAHLSLRGLPVCAFSSAGPCALRFTSARRMETTVNTHQVLPKVLHKRTLGLSAMSTTDLEAYFKDCLFKDWEELGEEIRLKVFVLGGCRHKLVCSPAPCNFFTSA

SEQ ID NO: 31 - protein, 154 aa, hepatitis B virus
MAARLCCQLDPARDVLCLRPVGAESRGRPFSGPLGTLSSPSPSAVSSDHGAHLSLRGLPVCAFSSAGPCALRFTSARRMETTVNAHQFLPKVLHKRTLGLSVMSTTDLEAYFKDCLFKDWEELGEEIRLKVFVLGGCRHKLVCAPAPCNFFTSA

SEQ ID NO: 32 - protein, 154 aa, hepatitis B virus
MAARLCCQLDPARDVLCLRPVSAESCGRPVSGSLGDLSSPSPSAVPADHGAHLSLRGLPVCAFSSAGPCALRFTSARRMETTVNAHQILPKVLHKRTLGLSAMSTTDLEAYFKDCLFKDWEELGEEIRLKVFVLGGCRHKLVCAPAPCNFFTSA

SEQ ID NO: 33 - protein, 134 aa, hepatitis B virus
MAARLCCQLDPTRDVLCLRPVGAESSGRSLSGSLGAVSPPSPSAVPADDGSHLSLRGLPVCSFSSAGPCALRFTSARRMETTVNAPRSLPTILHKRTLGLSGRSMTWIEEYIKDCVFKDWEELGEEIRLNIRRL

SEQ ID NO: 34 - protein, 154 aa, hepatitis B virus
MAARLCCQLDPSRDVLCLRPVSAESSGRPLPGPFGALSPPSPSAVPADHGAHLSLRGLPVCSFSSAGPCALRFTSARYMETAMNTSHHLPRQLYKRTLGLFVMSTTGVEKYFKDCVFAEWEELGNESRLMTFVLGGCRHKLVCAPAPCNFFTSA

SEQ ID NO: 35 - protein, 155 aa, hepatitis B virus
MAARLCCQLDPARDVLCLRPVGAESCGRPLSWSLGALPPSSPPRVPADDGSHLSLRGLPACAFSSAGPCALRFTSARRMETPVNAPWNLPTTLHKRTLGLLCNRSTTWIEEYIKDCVFKDWEESGEELRLMVYVLGGCRHKLVCSPAPCHFFTSA

SEQ ID NO: 36 - DNA, 465 bp, hepatitis B virus
atggctgctagggtgtgctgccaactggatcctgcgcgggacgtcctttgtctacgtcccgtcggcgctgaatcccgcggacgacccgtctcggggccgtttgggcctctaccgtccccttcttcatctgccgttccggccgaccacggggcgcacctctctttacgcggtctccccgtctgtgccttctcatctgccggaccgtgtgcacttcgcttcacctctgcacgtcgcatggaaaccaccgtgaacacccaccaggtcttgcccaaggtcttacataagaggactcttggactctcagcaatgtcaacaaccgaccttgaggcatacttcaaagactgtttgtttaaagactgggaggagttgggggaggagattaggttaaaggtctttgtactaggaggctgtaggcataaattggtctgttcaccagcaccatgcaactttttcacctctgcctaa

SEQ ID NO: 37 - DNA, 465 bp, Artificial
  synthetic
atggccgccagagtgtgctgtcagctggaccctgctagggacgtgctgtgcctgaggcctgtgggagccgagtctagaggcagacctgtgtccggccctttcggccctctgcctagcccttcttcttctgccgtgcctgccgatcacggcgcccacctgtctctgagaggactgcctgtgtgcgccttcagctctgccggaccttgcgccctgagattcaccagcgccagacggatggaaaccaccgtgaacacccaccaggtgctgcccaaggtgctgcacaaaagaaccctgggcctgagcgccatgagcaccaccgatctggaagcctacttcaaggattgcctgttcaaggactgggaggaactgggcgaagagatccggctgaaggtgttcgtgctgggcggctgcagacacaagctcgtgtgtagccctgccccctgcaacttcttcacctccgcctga

SEQ ID NO: 38 - DNA, 20 bp, Artificial
  primer
acaggtcggtttccaggttg

SEQ ID NO: 39 - DNA, 20 bp, Artificial
  primer
ggacatcaaggtggtgttca

(19) United States
(12) Patent Application Publication
(10) Pub. No.: US 2022/0202770 A1
(43) Pub. Date: Jun. 30, 2022

(54) COMPOSITION FOR TREATING HEPATITIS B, AND METHOD FOR EVALUATING REPLICATION ACTIVITY OF HEPATITIS B VIRUS

(71) Applicant: RIKEN, Wako-shi (JP)

(72) Inventor: Kenji OGAWA, Wako-shi (JP)

(73) Assignee: RIKEN, Wako-shi (JP)

(21) Appl. No.: 17/655,258

(22) Filed: Mar. 17, 2022

Related U.S. Application Data
(62) Division of application No. 16/323,720, filed on Feb. 6, 2019.

(30) Foreign Application Priority Data
Aug. 10, 2016 (JP) 2016-158252

(51) Int. Cl. A61K 31/365; A61K 31/352; A61K 31/519; A61P 1/16; A61P 31/20; C12N 15/09; A61K 31/16; C12N 9/99; C12Q 1/6897; C12N 7/00; A61K 31/164; C12N 15/85

(52) U.S. Cl. CPC A61K 31/365; A61K 31/352; A61K 31/519; A61P 1/16; A61P 31/20; C12N 15/09; C12N 2730/10143; C12N 9/99; C12Q 1/6897; C12N 7/00; A61K 31/164; C12N 15/85; A61K 31/16

(57) ABSTRACT

An evaluation system for replication activity of HBV capable of visualizing and quantifying replication of HBV DNA in a short period of time inexpensively, safely, and rapidly and a method for evaluation using the system are developed and provided. Moreover, a novel composition for inhibiting HBV replication with a mode of action different from that of conventional anti-HBV drugs is developed and provided. A therapeutic agent for hepatitis B comprising as an active ingredient an HBV-Pol activity inhibitor consisting of a phosphorylation inhibitor that inhibits phosphorylation of a TxY motif present in Terminal protein region of HBV-Pol is provided.

### CROSS-REFERENCE TO RELATED APPLICATIONS

This application is a divisional of U.S. application Ser. No. 16/323,720, filed Feb. 6, 2019, which in turn is a 371 of PCT/JP2017/029203, filed Aug. 10, 2017, which claims benefit of Japanese Patent Application No. 2016-158252 filed on Aug. 10, 2016, the contents of each of which are incorporated herein by reference.

### Reference to a Sequence Listing Submitted Electronically Via EFS-Web

The content of the electronically submitted sequence listing, file name: 522-1147-D_SeqListing.txt; size: 97,020 bytes; and date of creation: Mar. 17, 2022, filed herewith, is incorporated herein by reference in its entirety.

### TECHNICAL FIELD

The present invention relates to a composition for treating hepatitis B, comprising a MAP kinase inhibitor as an active ingredient, an evaluation system for replication activity in hepatitis B virus and a method for evaluating replication activity using the system.

### BACKGROUND ART

Hepatitis B is viral hepatitis that develops by infection of hepatitis B virus (herein, often referred to as “HBV”). Hepatitis B is transmitted via the blood and the body fluid of HBV infected persons and main routes of infection are therefore known to be vertical transmission (mother-to-child transmission) by which children are infected at birth via the blood of HBV infected mothers and horizontal transmission by sexual contact, tattooing, blood transfusion, reuse of syringe or needle stick accidents in mass vaccination, or the like (Non Patent Literature 1).

HBV infections are classified roughly into transient infection and chronic infection. HBV infection at the age of 5 years or older does not usually establish chronic infection but ends up as transient infection since immunocompetence is sufficiently developed. Then, infected persons acquire permanent immunity. However, about 20-30% develop acute hepatitis B and it is estimated that 1% of them develops fulminant hepatitis. Meanwhile, persons with chronic infection are mostly those infected by mother-to-child transmission, but chronic infection may be otherwise established when persons at the age of 3 years or younger, whose immune system is immature, are infected by causes such as medical practice or intrafamilial infection. Patients with HBV chronic infection are called HBV carriers and 10-15% of them develop chronic hepatitis, which may progress to liver cirrhosis or liver cancer. The number of HBV carriers is estimated to be 1.5 million in Japan and 300 to 400 million all over the world (Non Patent Literature 2).

Currently, nucleotide/nucleoside analog formulations represented by Lamivudine and Entecavir are frequently used as therapeutic agents for chronic hepatitis B. These therapeutic agents have achieved certain results in decreasing the blood virus load and in being capable of delaying development or progress of liver cirrhosis or hepatocellular cancer. However, since these agents cannot remove HBV DNA in hepatocytes, stopping the administration of the agents increases blood HBV DNA again and results in recurrence of hepatitis. Therefore, the long-term administration of a therapeutic agent is necessary. Moreover, the recurrence during the long-term treatment using one of the aforementioned nucleotide/nucleoside analog formulations is associated with the emergence of drug-resistant virus. This makes treatment of chronic hepatitis B even more difficult (Non-Patent Literature 3 and 4).

By the reasons described above, development of a novel HBV therapeutic agent with a mode of action different from that of conventional nucleotide/nucleoside analog formulations is desired. Development of such a therapeutic agent requires an experiment system that makes it possible to quantify and evaluate HBV infection and HBV replication accurately. There is a plurality of evaluation systems for analyzing HBV infection and HBV replication, but any of the systems has a safety problem and/or a problem that it is difficult to handle a large quantity of samples since infectious virus is used in these systems. Furthermore, cells that can be efficiently infected with HBV currently are limited to primary human hepatocyte culture systems or expensive HepaRG (R) cells and this largely limits available cells. Moreover, even the cells overexpressing NTCP, which is considered to be a receptor of HBV, are infected at a very low efficiency, though they are infected and the detection of the replication requires culturing for a long period of time, such as 7 to 12 days even when using an established cell line (such as HepG2.2.15 or HepAD38) in which HBV genome is inserted. Therefore, there is a big problem that these systems have low throughput. Such problems of HBV infection experiments make discovery of novel drugs for HBV difficult.

### CITATION LIST

### Non Patent Literature

Non Patent Literature 1:
Aspinall E. J. et al., 2011, Occup Med (Lond), 61: 531-540.
Non Patent Literature 2:
Fattovich G., et al., 2008, J Hepatol, 48: 335-352.
Non Patent Literature 3:
Lau D. T. et al., 2000, Hepatology, 32: 828-834.
Non Patent Literature 4:
Koumbi L., 2015, World J Hepatol, 7: 1030-1040.

### SUMMARY OF INVENTION

### Technical Problem

An object of the present invention is to develop and provide an evaluation system for replication activity of HBV capable of visualizing and quantifying replication of HBV DNA in a short period of time inexpensively, safely, and rapidly using common cells but not using infectious virus and a method for evaluation using the system.

Another object of the present invention is to isolate a novel anti-HBV compound with a mode of action different from that of conventional anti-HBV agents by applying the evaluation system for HBV replication activity to a method for screening and to provide the compound as a therapeutic agent for hepatitis B effective for recurrent hepatitis B.

### Solution to Problem

To achieve the aforementioned objects, the present inventors have visualized replication of HBV genome in a short period of time using common cells but not using infectious virus and constructed an evaluation system for HBV replication activity capable of quantifying the activity.

Moreover, search for natural anti-HBV compounds in a filamentous fungus extract library using this evaluation system for HBV replication activity resulted in obtaining Hypothemycin as a compound that inhibits replication of HBV in a concentration-dependent manner. Hypothemycin is an inhibitor of MAPK (mitogen-activated protein kinase) kinase (herein, often referred to as “MAPKK”) that contributes to the MAPK pathway. It was not conventionally known at all that any MAPK kinase inhibitor has the HBV replication inhibitory activity. Therefore, the present inventors examined 4 known MAPK kinase inhibitors for the HBV replication inhibitory activity and the examination has revealed that all the MAPK kinase inhibitors have the activity. Moreover, overexpression of constitutively active MEK-1 in host cells infected with HBV markedly promoted HBV replication. This result indicated that the MAPK kinase activity in host cells has an important role in replication of HBV. Furthermore, the analysis of the amino acid sequence of proteins derived from HBV has revealed the presence of TxY (T denotes a threonine residue, x denotes any amino acid residue, and Y denotes a tyrosine residue), which is a phosphorylation motif of MAPK kinase, in Terminal protein region of HBV-Pol in any HBV genotype. Therefore, it was considered that MAPK kinase phosphorylates the phosphorylation motif and activates HBV-Pol, which led to the supposition that replication of HBV may be inhibited by inactivation of HBV-Pol by MAPK kinase inhibitor inhibiting phosphorylation of HBV-Pol by MAPK kinase.

The present invention is based on the new findings described above and provides the following:
(1) A method for inhibiting replication of HBV, comprising the step of inhibiting phosphorylation of a threonine residue (T) and a tyrosine residue (Y) in a TxY motif (x is any amino acid residue) present in Terminal protein region of HBV polymerase (HBV-Pol).
(2) A composition for inhibiting HBV replication, comprising a MAPK (mitogen-activated protein kinase) kinase inhibitor.
(3) A composition for inhibiting HBV replication according to (2), wherein the MAPK kinase inhibitor is any one or more selected from the group consisting of Hypothemycin represented by Formula (1), Trametinib represented by Formula (2), PD98059 represented by Formula (3), and PD184352 represented by Formula (4).

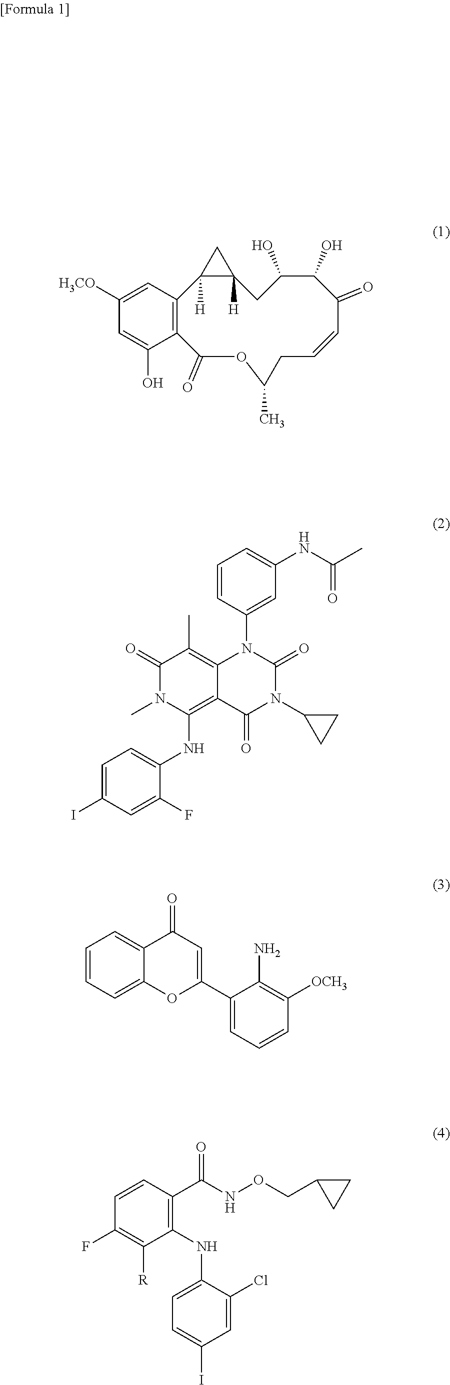

(4) A composition for treating hepatitis B, comprising a MAPK kinase inhibitor.
(5) The composition for treating hepatitis B according to (4), wherein the composition is used in combination with a nucleotide/nucleoside analog for treating hepatitis B.
(6) A nucleic acid comprising an epsilon sequence derived from HBV, wherein the nucleic acid comprises any one or more reporter sequences comprising an intron on the 3′ terminal side of the epsilon sequence.
(7) The nucleic acid according to (6), further comprising a Direct Repeat 1 sequence derived from hepatitis B virus on the 3′ terminal side of the epsilon, wherein the reporter sequence is comprised between the epsilon sequence and the Direct Repeat 1 sequences.
(8) The nucleic acid according to (6), comprising in this order from the 5′ terminal side: a first epsilon sequence derived from hepatitis B virus, a Direct Repeat 2 sequence, a Direct Repeat 1 sequence, and a second epsilon sequence, wherein the reporter sequence is comprised at any position between the sequences.
(9) A vector comprising a promoter capable of inducing gene expression in a host cell, and a nucleic acid according to any of (6) to (8) placed downstream of the promoter in a state that allows for the expression.
(10) A host cell transformed with a vector according to (9).
(11) An evaluation system for HBV replication activity for evaluating replication activity in HBV, comprising: a vector according to (9); an HBV-Pol expression vector in which a P gene from HBV is placed in a state that allows for the expression downstream of a promoter capable of inducing gene expression in a host cell; and an HBc expression vector in which a C gene from HBV is placed in a state that allows for the expression downstream of a promoter capable of inducing gene expression in a host cell.
(12) The evaluation system according to (11), further comprising an HBx expression vector in which an X gene from HBV is placed in a state that allows for the expression downstream of a promoter capable of inducing gene expression in a host cell.
(13) The evaluation system according to (11) or (12), further comprising a primer pair designed to be capable of detecting the reporter sequence from which the intron has been removed.
(14) A method for evaluating HBV replication activity, comprising: an introduction step of introducing into a host cell a vector according to (9), an HBV-Pol expression vector in which a P gene from HBV is placed in a state that allows for the expression downstream of a promoter capable of inducing gene expression in a host cell, and an HBc expression vector in which a C gene from HBV is placed in a state that allows for the expression downstream of a promoter capable of inducing gene expression in a host cell; a culturing step of culturing the host cell after the introduction step; an extraction step of extracting DNA from the host cell after the culturing step; and a detection step of detecting a gene product of the reporter sequence in the vector, wherein the gene product can be comprised in the DNA obtained in the extraction step and is the reporter sequence from which the intron has been removed.
(15) The method according to (14), further introducing into the host cell a HBx expression vector in which an X gene from HBV is placed in a state that allows for the expression downstream of the promoter capable of inducing gene expression in the host cell in the introduction step.

The specification of the present application incorporates the contents disclosed in JP Patent Application No. 2016-158252, to which the present application claims priority.

### Advantageous Effects of Invention

According to the method for evaluating HBV replication activity according to the present invention, it is possible to visualize replication of HBV genome in a short time and to quantify the activity inexpensively, safely, and rapidly using common cells but not using infectious virus. Moreover, use of the method for evaluating HBV replication activity according to the present invention as screening means makes it possible to search for an anti-HBV compound having HBV replication inhibitory activity.

The composition for inhibiting HBV replication or composition for treating hepatitis B comprising a MAPK kinase inhibitor according to the present invention makes it possible to provide a composition for inhibiting HBV replication or a composition for treating hepatitis B having a mode of action different from that of conventional nucleotide/nucleoside analogs for treating hepatitis B, and a method for inhibiting HBV replication.

Moreover, synergistic effect with an anti-HBV activity can be expected by using the composition for treating hepatitis B according to the present invention in combination with a conventional nucleotide/nucleoside analog for treating hepatitis B.

Furthermore, the composition for inhibiting HBV replication or composition for treating hepatitis B comprising a MAPK kinase inhibitor according to the present invention can be an effective therapeutic agent for hepatitis B since the composition also exhibits anti-HBV activity to drug-resistant HBV.

### BRIEF DESCRIPTION OF DRAWINGS

FIG. 1A is a schematic diagram illustrating the structure of genomic DNA of HBV. The portion represented by the black bold line in the center is about 3.2 Kb of relaxed circular double stranded genomic DNA (rcDNA) of HBV. The gray bold lines are ORFs (open reading frames) of the 4 genes encoded in the genomic DNA of HBV and C, P, S, and X respectively represent the positions of the C gene, the P gene, the S gene, and the X gene. The outer thin black line represents pregenomic RNA (herein, often referred to as “pgRNA”), which is the longest 3.5 kb mRNA among the mRNAs synthesized from the template (−) strand of HBV genomic DNA. FIG. 1B is a schematic diagram illustrating the structure of pgRNA. In the figure, DR1 and DR2 respectively denote Direct repeat 1 and 2 sequences and c denotes the ε sequence. The nucleic acid for evaluating HBV replication activity (evaluation nucleic acid for HBV replication activity) according to the present invention was produced based on the nucleotide sequence of this pgRNA.

FIG. 2A is a schematic diagram illustrating an example of the evaluation nucleic acid for HBV replication activity according to the present invention. FIG. 2B is a schematic diagram illustrating a reporter pgRNA when an evaluation vector for HBV replication activity in which the evaluation nucleic acid for HBV replication activity illustrated in A is incorporated in an expression vector is introduced into cells and expressed. In the reporter pgRNA, the intron in the reporter sequence is removed by pre-mRNA splicing.

FIG. 2C is a schematic diagram illustrating reporter minus strand DNA (reporter (−) DNA) synthesized by the reverse transcription activity of HBV-Pol from the reporter pgRNA illustrated in B as template. The evaluation nucleic acid for HBV replication activity and the reporter minus strand DNA can be distinguished by the presence or absence of the intron.

FIG. 3 is a schematic diagram illustrating an example of expression vectors constituting the evaluation system for HBV replication activity according to the present invention. FIG. 3A is a schematic diagram of pBB-intron, which is an example of the evaluation vector for HBV replication activity according to the present invention. FIG. 3B is a schematic diagram of pCI—HBV-Pol, which is an expression vector for the P gene, which encodes HBV-Pol. FIG. 3C is a schematic diagram of pCI-HBc, which is an expression vector for the C gene, which encodes HBc. FIG. 3D is a schematic diagram of pCI-HBx, which is an expression vector for the X gene, which encodes HBx.

FIG. 4A illustrates a result of agarose gel electrophoresis. The figure illustrates the PCR product (131 bp) when DNA is extracted and subjected to PCR with a primer pair that specifically detects the reporter minus strand DNA after HeLa cells are transfected with the evaluation vector for HBV replication activity together with the indicated combination of HBV protein expression vectors (C: HBc, P: HBV-Pol, X: HBx). FIG. 4B illustrates a standard curve for reporter minus strand DNA. The standard curve was made by diluting the DNA extracted from the cells by serial dilution and using real-time PCR. FIG. 4C illustrates a result obtained by performing real-time PCR with the DNA sample used in the experiment for FIG. 4A and quantitative analysis with the standard curve in FIG. 4B. FIG. 4D illustrates a result of quantitative analysis of the correlation between the expression levels of the HBV proteins (HBc, HBV-Pol, and HBx) and DNA replication by real-time PCR.

FIG. 5 illustrates the HBV DNA replication-inhibiting effect of Entecavir determined using the method for evaluating HBV replication activity according to the present invention.

FIG. 6 illustrates the effect of a natural compound isolated from a library of filamentous fungus culture extracts on HBV DNA replication, the amount of genomic DNA (G3PDH exon 8), and the amount of mitochondrial DNA (ND5).

FIG. 7 illustrates the HBV DNA replication-inhibiting effect of MAPK kinase inhibitors. The figure illustrates the effects of 4 different MAPK kinase inhibitors (PD98059, PD184352, Trametinib, Hypothemycin) and Entecavir, which is an anti-HBV agent, as a positive control on HBV DNA replication (n=3, * P<0.01, ** P<0.001, *** P<0.00001).

FIG. 8 illustrates the effect of expression of various MEK1 on HBV DNA replication, the amount of genomic DNA (G3PDH exon 8), the amount of mitochondrial DNA (ND5), pgRNA, and host mRNA transcription when the expression vectors for the wild type (Wt), constitutively active (2E and 2D), and dominant negative mutant (2A) human MEK1 are individually introduced into HeLa cells, using the method for evaluating HBV replication activity according to the present invention.

FIG. 9A is a schematic diagram illustrating the regions of HBV-Pol in Genotype C (HBV/C-Pol). The position numbers shown in the FIG. 9A corresponds to the amino acid position of SEQ ID NO: 3, which is the amino acid sequence of HBV/C-Pol. FIGS. 9B and C illustrate the comparison of nucleotide sequences of the putative TxY motif in HBV-Pol in Genotypes A to H. The numbers shown in the FIGS. 9B and C correspond to the amino acid position of the amino acid sequence of each genotype of HBV-pol. The amino acid sequence of genotypes A, B, C, D, E, F, and H are shown in SEQ ID NOs: 1, 2, 3, 4, 5, 6, 7, and 8, respectively. The TxY motif (TKY) in Terminal protein region is conserved in all of Genotypes A to H, but the TxY motif (²⁸⁴TAY²⁸⁶) in Spacer region is only found in Genotype C.

FIG. 10A is a schematic diagram illustrating non-phosphorylated mutant HBV/C-Pol based on HBV/C-Pol in Genotype C. A number range for the amino acid sequence shown in the FIG. 10A corresponds to the number of SEQ ID NO: 3. FIG. 10B illustrates the activity of non-phosphorylated mutant HBV/C-Pol for promoting HBV replication by the forced expression of constitutively active MEK.

FIG. 11 illustrates synergistic effect of the combination of Hypothemycin and Entecavir on HBV DNA replication inhibiting activity.

FIG. 12-1 is a schematic diagram illustrating a region of reverse transcription enzyme (RT) of drug-resistant HBV/C-Pol shown in SEQ ID NO:3. The numbers shown in FIG. 12-1 correspond to the amino acid position from the first amino acid of the RT region. As shown in FIG. 9A, since a start position of the RT region in HBV-Pol is position 347, the positions 180, 184, 202, and 204 in the RT region correspond to the positions 526 (=346+180), 530 (=346+184), 548 (=346+202), 550 (=346+204) of SEQ ID NO:3, respectively. L, T, S, M, I, and G respectively represent leucine, threonine, serine, methionine, isoleucine, and glycine.

FIG. 12-2 illustrates a result on the inhibiting effect of MAPK kinase inhibitors on the HBV replication activity of the drug-resistant HBV-Pol.

FIG. 13 illustrates the effect on reverse transcription of a reporter pgRNA when HeLa cells were transfected with various deleted forms of evaluation vectors for HBV replication activity together with the HBV protein expression vector CP (C: HBc, P: HBV-Pol) or CPX (C: HBc, P: HBV-Pol, X: HBx). In the figure, Δε1, ΔDR2, ΔDR1(2), and Δε2 respectively represent transfection of HeLa cells with pBB-intron (→ε1), pBB-intron (ΔDR2), pBB-intron (ΔDR1(2)), and pBB-intron (Δε2) as ΔpBB-intron, a deleted form of evaluation vector for HBV replication activity.

### DESCRIPTION OF EMBODIMENTS

### 1. Hepatitis B Virus Polymerase Activity Inhibitor (HBV-Pol Activity Inhibitor)

### 1-1. Summary

The first aspect of the present invention is a hepatitis B virus polymerase (herein, often referred to as “HBV-Pol”) activity inhibitor. The HBV-Pol inhibitor according to the present invention consists of a phosphorylation inhibitor that inhibits phosphorylation of an activation site in HBV-Pol. The HBV-Pol inhibitor according to the present invention makes it possible to inhibit replication of HBV by inhibiting HBV-Pol activity.

### 1-2. Definition

“Hepatitis B virus (HBV)” is a DNA virus in the genus Orthohepadnavirus in the family Hepadnaviridae and the causative virus of hepatitis B. 8 genotypes (Genotypes A, B, C, D, E, F, G, and H) of HBV based on the difference of the gene sequence are known. These genotypes are different in geographical distribution and/or pathology. For example, in Japan, most of infected persons have been infected with virus having Genotype C (herein, often referred to as “HBV/C”. Other genotypes are referred to similarly.) and HBV/B-infected persons have been the second most, though HBV/A infected persons have been increasing in recent years. In Europe and the United States, many infected persons are infected with HBV/A or HBV/D. While it is known that about 20 to 30% of persons that have developed acute hepatitis in HBV/A infection develop chronic hepatitis, a small percent of persons that have developed acute hepatitis in HBV/B or HBV/C infection develop chronic hepatitis.

“Hepatitis B virus polymerase” (HBV polymerase: HBV-Pol, or HBV-DNA Pol) is a DNA polymerase having reverse transcriptase activity and being encoded by the P gene in the HBV genome and involved in the replication of HBV (Summers J., & Mason W. S., 1982, Cell, 29: 403-415). Also the amino acid sequence of HBV-Pol slightly varies depending on the genotype. For example, HBV-Pol of HBV/A (herein, often referred to as “HBV/A-Pol”. Other genotypes are also referred to similarly.) consists of the amino acid sequence set forth in SEQ ID NO: 1, HBV/B-Pol consists of the amino acid sequence set forth in SEQ ID NO: 2, HBV/C-Pol consists of the amino acid sequence set forth in SEQ ID NO: 3, HBV/D-Pol consists of the amino acid sequence set forth in SEQ ID NO: 4, HBV/E-Pol consists of the amino acid sequence set forth in SEQ ID NO: 5, HBV/F-Pol consists of the amino acid sequence set forth in SEQ ID NO: 6, HBV/G-Pol consists of the amino acid sequence set forth in SEQ ID NO: 7, and HBV/H-Pol consists of the amino acid sequence set forth in SEQ ID NO: 8.

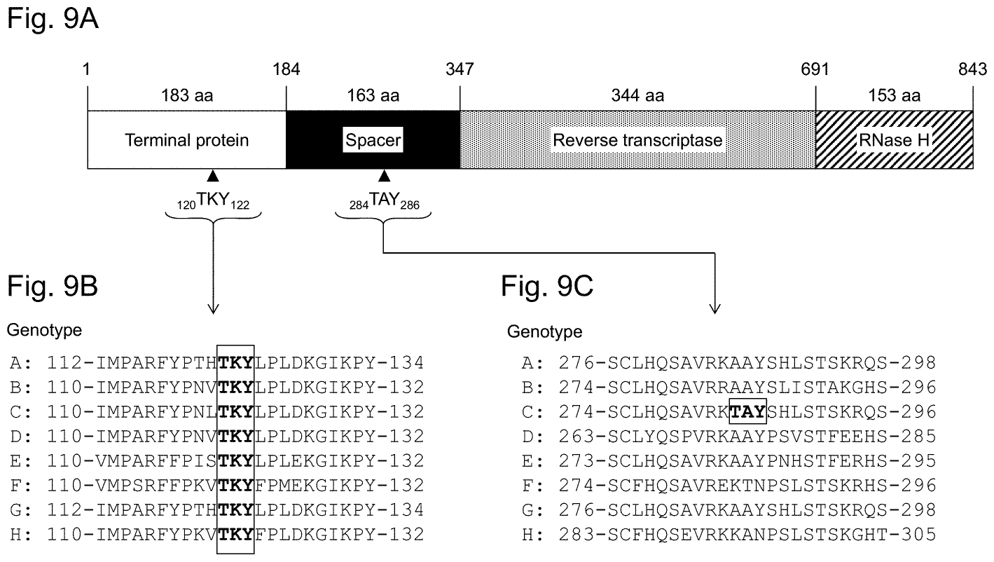

HBV-Pol contains 3 functionally essential regions (Terminal protein region, Reverse transcriptase region, RNaseH region) in the amino acid sequence (FIG. 9A).

“Terminal protein region” locates in the N-terminal side in HBV-Pol and is composed of, for example, 183 amino acids in Genotype C. This region binds to the vicinity of Direct repeat 1 (DR1) involved in the virus replication and considered to function as a primer in reverse transcription (Bartenschlager R. & Schaller H., 1988, EMBO J., 7: 4185-4192). Terminal protein region contains a TxY motif, which is an activation site of HBV-Pol as described below.

“Reverse transcriptase region” locates on the C-terminal side of Terminal protein region and is composed of, for example, 344 amino acids in Genotype C. The region is a region that shows high homology with amino acid sequences of reverse transcriptases in retroviruses and the (−) strand of HBV DNA is synthesized in HBV replication by reverse transcription activity of this region using as template pregenomic RNA transcribed from HBV genome. The (+) strand of HBV DNA is further synthesized by DNA polymerase activity using this (−) strand as template.

“RNase H region” locates in the C-terminal side in HBV-Pol and is composed of, for example, 153 amino acids in Genotype C. This region is a region having activity that degrades pregenomic RNA used as template for reverse transcription in the replication of HBV DNA.

Besides the 3 regions described above, Spacer region is present between Terminal protein region and Reverse transcriptase region. This region is not necessary for the function of HBV-Pol.

### 1-3. Constitution

The HBV-Pol activity inhibitor according to the present invention consists of a phosphorylation inhibitor that inhibits the phosphorylation of the activation site in HBV-Pol.

The activation site in HBV-Pol is the TxY motif in Terminal protein region in HBV-Pol. “TxY motif” is a conserved sequence consisting of 3 amino acid residues: a T (threonine) residue, x (any amino acid residue, whose examples include amino acid residues such as lysine [K], glutamic acid [E], proline [P], and glycine [G]), and a Y (tyrosine) residue. In HBV-Pol, for example, in Genotype C, the motif locates at the positions 120 to 122 when the position 1 is defined to be the start methionine (FIGS. 9A and B). The HBV-Pol in Genotype C has also one TxY motif-like sequence at the positions 284 to 286 in Spacer region, besides that in Terminal protein region (FIGS. 9A and C). However, the results of Examples described below indicate that this sequence is neither phosphorylated nor involved directly in the activity of HBV-Pol. Therefore, it is considered that the sequence is not an activation site.

HBV-Pol is considered to be activated by the phosphorylation of the T residue and the Y residue in the TxY motif in Terminal protein region and function as a DNA polymerase in the replication of HBV. Therefore, the HBV-Pol activity inhibitor according to the present invention may be a substance that acts to inhibit the phosphorylation (phosphorylation inhibitor) of the T residue and the Y residue in the TxY motif. Examples of such a phosphorylation inhibitor include substances that inhibit the catalytic activity of the kinase that phosphorylates the TxY motif and substances that bind to the TxY motif to inhibit the binding between the kinase and the TxY motif.

The kinase to be targeted by the HBV-Pol activity inhibitor may be any type of inhibitor, as long as it can phosphorylate the T residue and/or the Y residue in the TxY motif. In general, the TxY motif is known as an activation site of the MAPK (Mitogen-Activated Protein Kinase) family such as ERK (Extracellular signal-Regulated Kinase), p38, and JNK (c-Jun N-terminal Kinase). The factors that phosphorylate the TxY motifs in this MAPK and activate MAPK are MAPK kinases (MAPKKs). The MAPK kinases include MEK1 (MAPKK1), MEK2 (MAPKK2), MKK3 (MAPKK3), MKK4 (MAPKK4), MKK5 (MAPKK5), MKK6 (MAPKK6), and MKK7 (MAPKK7). As shown in Examples described below, it is considered that HBV-Pol is also phosphorylated by MAPK kinases at the TxY motif to be activated. Therefore, the MAPK kinases may be the target kinase of the HBV-Pol activity inhibitor according to the present invention.

When the HBV-Pol activity inhibitor according to the present invention is composed of a kinase inhibitor that inhibits the catalytic activity of the target kinase, the kinase inhibitor may be a MAPK kinase inhibitor. Examples of the MAPK kinase inhibitor include Hypothemycin represented by Formula (1), Trametinib represented by Formula (2), PD98059 represented by Formula (3), PD184352 represented by Formula (4), and U-126 represented by Formula (5) illustrated below.

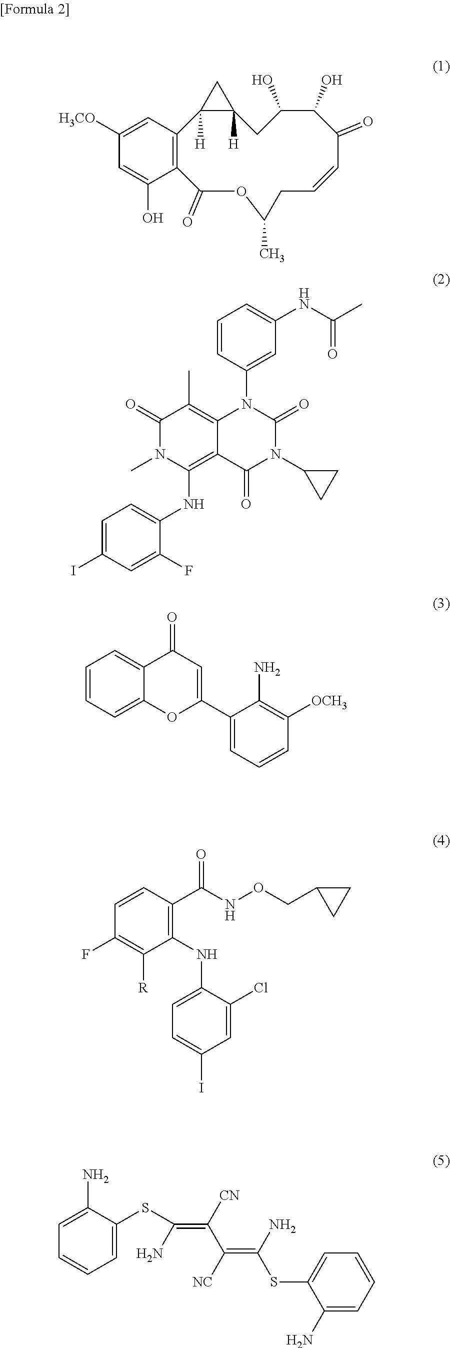

### 2. Method for Inhibiting Hepatitis B Virus Replication (Method for Inhibiting HBV Replication)

The second aspect of the present invention is a method for inhibiting hepatitis B virus replication (method for inhibiting HBV replication). The method according to the present invention comprises the step (inhibition step) of inhibiting the phosphorylation of the threonine residue and the tyrosine residue of the TxY motif present in Terminal protein region in HBV-Pol.

The aforementioned inhibition step is a step of administering a substance capable of inhibiting the phosphorylation of the threonine residue and the tyrosine residue in the aforementioned TxY motif to cells infected with HBV. Specific examples of such a substance include the HBV-Pol activity inhibitor according to the first aspect. In particular, the MAPK kinase inhibitor is preferred. Examples include the known MAPK kinase inhibitors represented by Formula (1) to Formula (5) illustrated above.

HBV-Pol is considered to be activated by the phosphorylation of the threonine residue and the tyrosine residue in the TxY motif present in Terminal protein region, as described above. Since HBV-Pol is involved in the replication of HBV, the inhibition of phosphorylation of both amino acid residues in the TxY motif results in the inhibition of the replication of HBV. The present invention is a method for inhibiting HBV replication based on such a mechanism.

3. Composition for Inhibiting Replication of Hepatitis B Virus (Composition for Inhibiting HBV replication)

### 3-1. Summary

The third aspect of the present invention is a composition for inhibiting replication of hepatitis B virus (composition for inhibiting HBV replication). The composition for inhibiting HBV replication according to the present invention comprises an HBV-Pol activity inhibitor according to the first aspect, in particular a MAP kinase inhibitor as an essential active ingredient and can inhibit the replication of genomic DNA of HBV to suppress the proliferation of HBV. Therefore, the composition can be used as an anti-HBV composition.

### 3-2. Constitution

### 3-2-1. Components

The components of the composition for inhibiting HBV replication according to the present invention will be described.

### (1) Active Ingredient

The composition for inhibiting HBV replication according to the present invention comprises the HBV-Pol activity inhibitor according to the first aspect, in particular a MAPK kinase inhibitor as an essential active ingredient.

The active ingredient may be composed of only the HBV-Pol activity inhibitor according to the first aspect, but the composition may be a combination composition comprising a combination of 2 or more other known anti-HBV agents. Moreover, the HBV-Pol activity inhibitor according to the first aspect may be composed of 1 inhibitor, but may comprise a plurality of inhibitors, for example, the 2 inhibitors Hypothemycin and Trametinib.

The aforementioned known anti-HBV agents are not particularly limited, but nucleotide/nucleoside analogs for treating hepatitis B are preferred. Examples of the nucleotide/nucleoside analogs for treating hepatitis B include Entecavir (ETV), Lamivudine (LAM), Adefovir, Tenofovir, Telbivudine, and clevudine. Any of these is a composition for inhibiting HBV replications that inhibits the reverse transcriptase activity of HBV. Since the HBV-Pol activity inhibitor according to the first aspect has a mode of action different from that of the nucleotide/nucleoside analogs for treating hepatitis B such as Entecavir and Lamivudine as shown in Example 8 described below, a synergistic inhibiting effect on the HBV replication can be obtained by the combination thereof.

The content of the active ingredient contained in the composition for inhibiting HBV replication is not particularly limited. In general, the content varies depending on the kind of the active ingredient, the dosage form, and the kind of the solvent or carrier, which are other components described below. Therefore, the content may be appropriately determined in consideration of each condition. The content may be determined such that an effective amount of the active ingredient is contained in a single dose of the composition for inhibiting HBV replication. However, when it is necessary to administer a large amount of the composition for inhibiting HBV replication to a subject to obtain the pharmacologic effect of the active ingredient, the amount may be divided into several doses to be administered for the reduction of the burden on the subject. In such a case, the amount of the active ingredient may comprise an effective amount in total. “The effective amount” refers to an amount that is necessary for functioning as an active ingredient and has little or no adverse side effects to the subject to which it is administered. This effective amount may vary depending on various conditions such as information of the subject, the route of administration, and the number of dosing. Therefore, when the composition for inhibiting HBV replication is used as a medicament, the final determination of the content of the active ingredient is made by the decision of a physician, a pharmacist, or the like.

As used herein, the “subject” refers to an object to which the HBV-Pol inhibitor according to the first aspect, the composition for inhibiting HBV replication according to this aspect, or the composition for treating hepatitis B according to the fourth aspect is administered. The subject is, for example, a cell (including a cultured cell), tissue, an organ, or an individual. When the subject is an individual, it is preferably a human individual and particularly preferably a hepatitis B infected person.

As used herein, the “information of the subject” is various information about characteristics and conditions of the subject. Examples of the information of the subject when the subject is a human individual include age, body weight, sex, general health condition, the presence or absence of a disease, the progression or severity of the disease, drug sensitivity, the presence or absence of combined drugs, and resistance to treatment.

### (2) Solvent

The composition for inhibiting HBV replication according to the present invention may be dissolved in a pharmaceutically acceptable solvent as needed. The “pharmaceutically acceptable solvent” refers to a solvent usually used in the art of drug formulation. Examples thereof include water or aqueous solutions or organic solvents. Examples of the aqueous solutions include physiological saline, isotonic solutions containing glucose or another pharmaceutic aid, phosphate buffer solutions, and sodium acetate buffer solutions. Examples of the pharmaceutic aid include D-sorbitol, D-mannose, D-mannitol, and sodium chloride as well as a non-ionic surfactant at a low-concentration, and a polyoxyethylene sorbitan fatty acid ester. Examples of the organic solvents include ethanol.

### (3) Carrier

The composition for inhibiting HBV replication according to the present invention may comprise a pharmaceutically acceptable carrier as needed. The “pharmaceutically acceptable carrier” refers to an additive usually used in the art of drug formulation. Examples thereof include an excipient, a binder, a disintegrator, a filler, an emulsifier, a fluidity improving agent, a lubricant, and human serum albumin.

Examples of the excipient include sugars such as monosaccharides, disaccharides, cyclodextrin and polysaccharides, metal salts, citric acid, tartaric acid, glycine, polyethyleneglycol, Pluronic, kaolin, silicic acid, or a combination thereof.

Examples the binder include starch paste made from plant starch, pectin, xanthan gum, simple syrup, a glucose solution, gelatin, tragacanth, methylcellulose, hydroxypropylmethylcellulose, sodium carboxymethylcellulose, shellac, paraffin, polyvinylpyrrolidone, or a combination thereof.

Examples of the disintegrator include the aforementioned starch, lactose, carboxymethyl starch, cross-linked polyvinylpyrrolidone, agar, laminaran powder, sodium hydrogen carbonate, calcium carbonate, alginic acid or sodium alginate, polyoxyethylene sorbitan fatty acid esters, sodium lauryl sulfate, stearic acid monoglyceride, or a salt thereof.

Examples of the filler include petrolatum, the aforementioned sugars, and/or calcium phosphate.

Examples of the emulsifier include sorbitan fatty acid esters, glycerin fatty acid esters, sucrose fatty acid esters, and propylene glycol fatty acid esters.

Examples of the fluidity improving agent and the lubricant include silicates, talc, stearates, or polyethyleneglycol.

The composition for inhibiting HBV replication according to the present invention may also comprise, in addition to those described above, a solubilization agent, a suspending agent, a diluent, a dispersant, a surfactant, a soothing agent, a stabilizer, an absorption enhancer, a bulking agent, a humectant, a moisturizer, a moistening agent, an adsorbent, a corrigent, a disintegration-suppressing agent, a coating, a colorant, a preservative, an antiseptic, an antioxidant, a flavor, a flavorant, an edulcorant, a buffer, an isotonizing agent, or the like usually used in pharmaceutical compositions or the like, as appropriate, if necessary.

The carrier is used to avoid or suppress the degradation of the aforementioned active ingredient by enzymes or the like in the subject as well as to facilitate the formulation and the mode of administration and to maintain the dosage form and the efficacy and may be used as appropriate as needed.

### 3-2-2. Dosage Form

The dosage form of the composition for inhibiting HBV replication according to the present invention is not particularly limited, as long as it is a form that allows the delivery to the site of interest without deactivating the active ingredient in the body of the subject.

The specific dosage form varies depending on the mode of administration described below. Since the mode of administration can be roughly classified into parenteral administration and oral administration, the composition may be formulated into a dosage form suitable for either of the modes of administration.

For example, if the mode of administration is parenteral administration, then a preferred dosage form is a liquid that can be administered directly to the target site or administered systemically through the circulatory system. Examples of the liquid include injections. The injections can be formulated by combining the active ingredient with the aforementioned excipient, emulsifier, suspension, surfactant, stabilizer, pH regulator, or the like as appropriate and mixing in a unit dose form required for a generally accepted pharmaceutical practice.

If the mode of administration is oral administration, then preferred dosage forms include solid formulations (including tablets, capsules, drops, and troches), granules, powdered medicine, powders and liquids (including liquids for internal use, emulsions, and syrups). Solid formulations may be formulated into dosage forms with a coating known in the art, for example, sugar coated tablets, gelatin coated tablets, enteric coated tablets, film coated tablets, double-coated tablets, or multilayered tablets, as needed.

Specific shapes and sizes of the aforementioned individual dosage forms are not particularly limited, as long as such shapes and sizes of the dosage forms are within the ranges of those of the dosage forms known in the art. Regarding the method of producing the composition for inhibiting HBV replication according to the present invention, the compound may be formulated according to a method usual in the art.

### 3-2-3. Mode of Administration

The mode of administration of the composition for inhibiting HBV replication according to the present invention may be oral administration or parenteral administration. In general, the oral administration is systemic administration, but the parenteral administration may be subdivided into local administration and systemic administration. Examples of the local administration include intramuscular administration, subcutaneous administration, tissue administration, and organ administration and the systemic administration includes cardiovascular administration, for example, intravenous administration (intravenous injection), intraarterial administration, and administration in the lymphatic vessel. When the composition for inhibiting HBV replication according to the present invention is administered locally, the compound may be administered directly to the liver by injection or the like. Moreover, when the composition is administered systemically, the compound may be administered into the circulatory organ by intravenous injection or the like. The dose may be an amount effective in having the effect of the active ingredient. The effective amount is appropriately selected depending on the information of the subject as described above.

Moreover, the composition for inhibiting HBV replication according to the present invention may be used in combination with 2 or more other known anti-HBV agents, in particular nucleotide/nucleoside analogs for treating hepatitis B.

### 4. Composition for Treating Hepatitis B

### 4-1. Summary

The fourth aspect of the present invention is a composition for treating hepatitis B. The composition for treating hepatitis B according to the present invention comprises the HBV-Pol activity inhibitor according to the first aspect, in particular a MAPK kinase inhibitor as an essential active ingredient and can suppress the proliferation of HBV by inhibiting the replication of HBV after the HBV infection to treat Hepatitis B.

As used herein, the “treatment” refers to the alleviation or elimination of symptoms associated with disease and/or the prevention or suppression of progression of disease and the healing of disease. As used herein, the “disease” means hepatitis B, unless otherwise specified.

### 4-2. Constitution

The third and fourth aspects differ only in their use. More specifically, while the composition for inhibiting HBV replication according to the third aspect is directed to the inhibition of replication of HBV in host cells, the composition for treating hepatitis B according to this aspect is directed to the treatment of hepatitis B infected person through the inhibition of replication of HBV. Therefore, the basic constitution of this aspect is substantially same as that described in “3-2. Constitution” as to the composition for inhibiting HBV replication according to the third aspect. Thus, the specific description is omitted here.

### 4-3. Method for Treating Hepatitis B

Hepatitis B can be treated by administering to a subject the composition for treating hepatitis B according to the present invention. A method for treating hepatitis B using such a composition for treating hepatitis B involves use of the activity of the HBV-Pol activity inhibitor according to the first aspect, in particular a MAP kinase inhibitor, which is an active ingredient of the composition for treating hepatitis B and therefore comprises the step (inhibition step) of inhibiting the phosphorylation of the threonine residue and the tyrosine residue of the TxY motif present in Terminal protein region in HBV-Pol, like the method for inhibiting HBV replication according to the aforementioned second aspect.

### 5. Hepatitis B Virus Polymerase Activity Promoter (HBV-Pol Activity Promoter)

### 5-1. Summary

The fifth aspect of the present invention is a hepatitis B virus polymerase activity promoter (HBV-Pol activity promoter). The HBV-Pol activity promoter according to this aspect has a reverse effect of the HBV-Pol activity inhibitor according to the first aspect and consists of a compound that phosphorylates the active site present in Terminal protein region in HBV-Pol.

HBV-Pol activity promoter according to the present invention can activate HBV-Pol and increase the amount of HBV replication in host cells by administering the promoter to cells infected with HBV.

5-2. Constitution The HBV-Pol activity promoter according to the present invention consists of a compound that phosphorylates the activation site, that is, the T residue and/or the Y residue in the TxY motif in HBV-Pol.

HBV-Pol is activated by the phosphorylation of the TxY motif in Terminal protein region. As described above, examples of the compound that phosphorylates the TxY motif include MAPK kinases. Therefore, the HBV-Pol activity promoter may be composed of a MAPK kinase. Specific examples of the MAPK kinase include MAPKK family members such as MEK1 (MAPKK1), MEK2 (MAPKK2), MKK3 (MAPKK3), MKK4 (MAPKK4), MKK5 (MAPKK5), MKK6 (MAPKK6), and MKK7 (MAPKK7). The MAPK kinase may be a constitutively active MAPK kinase in which a mutation is introduced. Examples thereof for human MEK1 include the constitutively active MEK1 in which serine (S) at the positions 218 and 222 is substituted with glutamic acid (E) and the constitutively active MEK1 in which serine (S) at the positions 218 and 222 is substituted with aspartic acid (D).

6. Hepatitis B virus replication activating agent

### 6-1. Summary

The sixth aspect of the present invention is a hepatitis B virus replication activating agent (HBV replication activating agent). The HBV replication activating agent according to this aspect comprises the HBV-Pol activity promoter according to the fifth aspect as an essential active ingredient and can promote the replication of HBV genomic DNA in host cells after HBV infection to enhance the proliferation. Therefore, the HBV replication activating agent according to the present invention has a reverse effect of the composition for inhibiting HBV replication according to the third aspect.

In general, the efficiency of HBV infection is very low. Cells that are efficiently infected with HBV are limited to cells from human hepatocyte primary culture systems or the HepaRG cell line and there is no established cell line that can be infected with HBV with an improved efficiency. This limitation of cells infected with HBV has been a cause of delaying the study on HBV infection and the development of compositions for treating hepatitis B. Use of the HBV replication activating agent according to the present invention makes it possible to improve the activity of HBV proliferation and the efficiency of HBV infection and can contribute to the study of HBV infection and the development of compositions for treating hepatitis B.

### 6-2. Constitution

The HBV replication activating agent according to the present invention comprises the HBV-Pol activity promoter according to the fifth aspect as an active ingredient.

The HBV-Pol activity promoter according to the fifth aspect may be the only active ingredient of the HBV replication activating agent or the agent may be a combination composition comprising a combination of 2 or more other known HBV proliferation promoters. Moreover, the HBV-Pol activity promoter according to the fifth aspect may be not only 1 promoter but comprise a plurality of promoters, for example, the 2 promoters MEK1 and MKK3.

The content of the active ingredient contained in the HBV replication activating agent according to the present invention is in accordance with the content of the active ingredient in the composition for inhibiting HBV replication according to the third aspect.

### 7. Evaluation Nucleic Acid for Hepatitis B Virus Replication Activity (Evaluation Nucleic Acid for HBV Replication Activity)

### 7-1. Summary

The seventh aspect of the present invention is a nucleic acid molecule that makes it possible to evaluate replication activity in hepatitis B virus (evaluation nucleic acid for HBV replication activity). The evaluation nucleic acid for HBV replication activity according to the present invention is a reporter gene that imitates the structure of the gene encoding pregenomic RNA (pgRNA) of HBV and constructed to allow quantification of the amount of minus strand DNA synthesized by the reverse transcription activity of HBV-Pol from template pgRNA.

### 7-2. Mechanism of HBV Replication

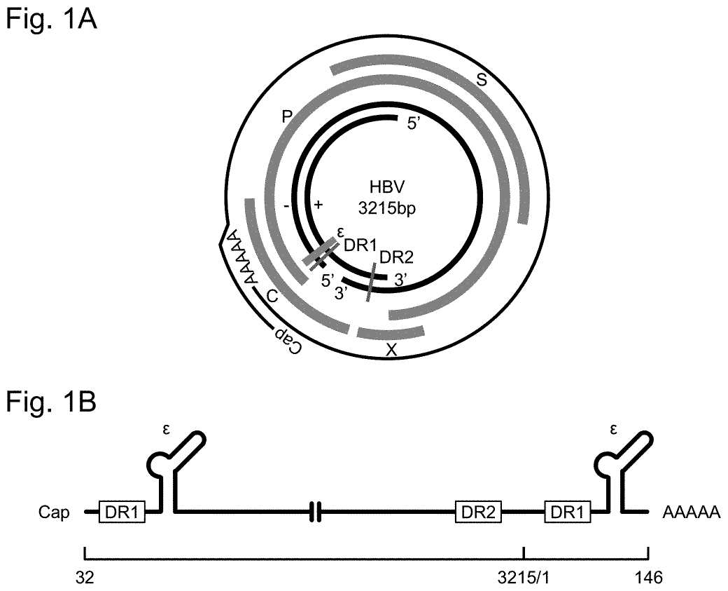

In the HBV genome, the plus strand ((+) strand) is shorter than the minus strand ((−) strand) as illustrated in FIG. 1A. Therefore, the HBV genome has an approximately 3.2 Kb relaxed circular DNA (rcDNA) having the single strand structure in part. HBV enters host hepatocytes via an unknown HBV-specific receptor and, after the establishment of infection, an endogenous DNA polymerase repairs the single stranded part in the nucleus of the host cell to produce covalently closed circular DNA (cccDNA). Using the (−) strand of this cccDNA as template, 4 mRNAs different in length (3.5 kb, 2.4 kb, 2.1 kb and 0.7 kb) are synthesized by RNA polymerase II (RNA pol II) derived from the host cell. Of these, the longest 3.5 kb mRNA is referred to as pregenomic RNA (pgRNA) and serves as the template of HBV genomic DNA.

The epsilon (RNA encapsidation signal epsilon; herein, often referred to as “ε”) sequence, which is an HBV capsid formation signal, is found at both 5′ and 3′ terminals of pgRNA, as illustrated in FIG. 1B. HBV-Pol interacts with this ε sequence and is incorporated into the nucleocapsid made of HBc. In the nucleocapsid, the (−) strand DNA of HBV is synthesized by the reverse transcription activity of Reverse transcription region in HBV-Pol and the primer function of Terminal protein region using the pgRNA as template. pgRNA after reverse-transcription is promptly degraded by the RNase H activity of RNase H region in HBV-Pol, leaving 17 nucleotides at the 5′ terminal.

The 5′ terminal of remaining pregenomic RNA after the synthesis of (−) strand DNA forms a pair with Direct Repeat 2 (herein, often referred to as “DR2”) in the (−) strand DNA. The synthesis of (+) strand DNA is started using this as a primer and (−) strand DNA as template. This synthesis stops before the completion and the nucleocapsid is enclosed in the envelope and released out of the cell.

### 7-3. Constitution

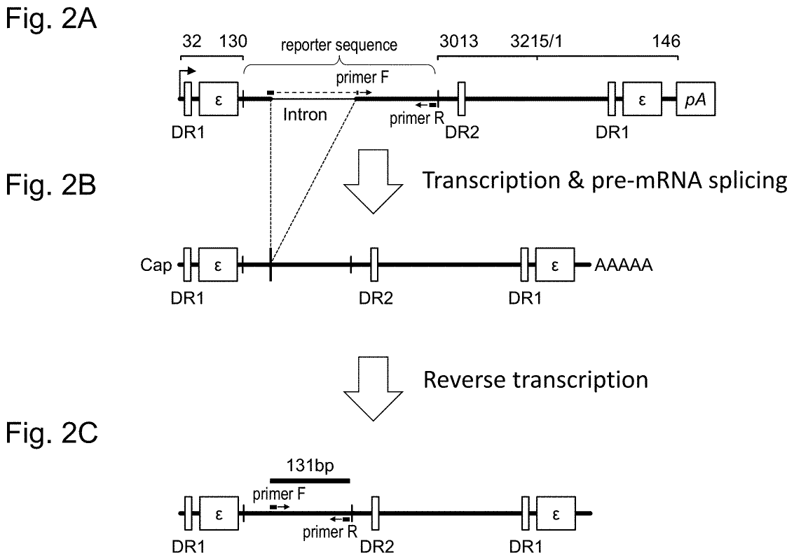

An example of the structure of the evaluation nucleic acid for HBV replication activity according to the present invention is illustrated in FIG. 2A. The evaluation nucleic acid for HBV replication activity is a gene encoding a reporter pgRNA (FIG. 2B) of HBV and the basic constitution thereof is based on the HBV genomic DNA. The evaluation nucleic acid for HBV replication activity according to the present invention comprises, as essential components, an epsilon sequence (ε sequence) derived from pgRNA of HBV and any reporter sequence being located on the 3′ terminal side of the epsilon sequence and comprising an intron. Moreover, the evaluation nucleic acid comprises a Direct Repeat 1 sequence derived from pgRNA of HBV as an optional component. Furthermore, the evaluation nucleic acid may comprise a Direct Repeat 2 sequence and/or another epsilon sequence as needed.

The “Direct Repeat 1 (herein, often referred to as “DR1”) sequence” is, for example, a sequence consisting of the 11 nucleotides set forth in SEQ ID NO: 9 in Genotype C and found at two positions in the 5′ terminal side and the 3′ terminal side on pgRNA, as illustrated in FIG. 1B. As used herein, the DR1 sequence in the 5′ terminal side and the DR1 sequence in the 3′ terminal side are respectively referred to as the first DR1 sequence and the second DR1 sequence.

The “Direct Repeat 2 (herein, often referred to as “DR2”) sequence” is a sequence consisting of the 11 nucleotides, same as the aforementioned DR1 sequence, set forth in SEQ ID NO: 10 and found between the first ε sequence described below and the aforementioned second DR1 sequence on pgRNA, as illustrated in FIG. 1B.

The “epsilon (ε) sequence” is found immediately downstream of the DR1 sequence on pgRNA, as illustrated in FIG. 1B. ε consists of a nucleotide sequence which is different depending on the genotype. Specifically, the epsilon sequence is the nucleotide sequence consisting of the 61 nucleotides set forth in SEQ ID NO: 11 in Genotype A, the nucleotide sequence consisting of the 61 nucleotides set forth in SEQ ID NO: 12 in Genotypes B to F, the nucleotide sequence consisting of the 61 nucleotides set forth in SEQ ID NO: 13 in Genotype G, and the nucleotide sequence consisting of the 61 nucleotides set forth in SEQ ID NO: 14 in Genotype H.

In pgRNA, the ε sequence forms a secondary structure consisting of the stem structure, the bulge structure, and the loop structure by self-folding as illustrated in FIG. 1B. As described above, the ε sequence functions as an HBV capsid formation signal and HBV-Pol interacts with this ε sequence and is incorporated into the nucleocapsid. As used herein, the c sequence located downstream of the first DR1 sequence and the ε sequence located downstream of the second DR1 sequence in pgRNA are respectively referred to as the first c sequence and the second ε sequence. Of these, the first ε sequence has been revealed by Examples of the present application to be essential for the HBV replication activity.

The DR1 sequence, the DR2 sequence, and the ε sequence in the evaluation nucleic acid for HBV replication activity may be derived from any genotype.

The “reporter sequence” is a unique sequence in the evaluation nucleic acid for HBV replication activity according to the present invention and has a structure comprising at least 1 intron removable by pre-mRNA splicing in any nucleotide sequence. The nucleotide sequence of the reporter sequence is not particularly limited. For example, the nucleotide sequence may be a nucleotide sequence derived from between the first ε sequence and the DR2 sequence in pgRNA or a nucleotide sequence derived from between the DR2 sequence and the first DR1 sequence in pgRNA or may be any gene sequence. Moreover, the length of the nucleotides comprising the intron is not particularly limited. For example, the length may be 150 nucleotides or more, 200 nucleotides or more, 250 nucleotides or more, 300 nucleotides or more, 350 nucleotides or more, or 400 nucleotides or more. The upper limit of the length is not limited, but the length may be 1000 nucleotides or less, 900 nucleotides or less, 800 nucleotides or less, 700 nucleotides or less, 600 nucleotides or less, or 500 nucleotides or less. The nucleotide length of the intron in the reporter sequence is also not particularly limited, but the length may be in the range of 30 nucleotides or more, 40 nucleotides or more, 50 nucleotides or more, 60 nucleotides or more, 70 nucleotides or more, 80 nucleotides or more, 90 nucleotides or more, 100 nucleotides or more, 110 nucleotides or more, 120 nucleotides or more, 130 nucleotides or more, 140 nucleotides or more, 150 nucleotides or more, 160 nucleotides or more, 170 nucleotides or more, 180 nucleotides or more, 190 nucleotides or more, or 200 nucleotides or more and 300 nucleotides or less, 290 nucleotides or less, 280 nucleotides or less, 270 nucleotides or less, 260 nucleotides or less, or 250 nucleotides or less.

The reporter pgRNA transcribed from the evaluation nucleic acid for HBV replication activity has an overall structure that imitates pgRNA, as illustrated in FIG. 2B. For example, when the reporter pgRNA comprises an ε sequence and a DR1 sequence, the sequences can be arranged in the order of the ε sequence (corresponding to the first c sequence) and the DR1 sequence (corresponding to the second DR1 sequence) from the 5′ terminal side. Moreover, when the reporter pgRNA comprises a DR2 sequence and another c sequence in addition to the ε sequence and the DR1 sequence, the sequences can be arranged in the order of the ε sequence (corresponding to the first ε sequence), the DR2 sequence, the DR1 sequence (corresponding to the second DR1 sequence) and another ε sequence (corresponding to the second ε sequence) from the 5′ terminal side. The position of the reporter sequence containing the intron is not particularly limited, but the sequence can be placed, for example, between the ε sequence and the DR1 sequence (corresponding to “the first c/second DR1 sequence”), between the ε sequence and the DR2 sequence (corresponding to “the first c/DR2 sequence”), and/or between the DR2 sequence and the DR1 sequence (corresponding to “the DR2/second DR1 sequence”). The first c/DR2 sequence or the DR2/second DR1 sequence other than the reporter sequence may comprise a spacer sequence. The nucleotide sequence of the spacer sequence is not particularly limited. Moreover, the spacer sequences between the sequences may be different in nucleotide sequence and in nucleotide length. A preferred spacer sequence is the nucleotide sequence present in the same position in pgRNA. For example, the second DR1/second ε sequence may be the same nucleotide sequence as the second DR1/second ε sequence in pgRNA. Moreover, the first c/DR2 sequence or the DR2/second DR1 sequence other than the reporter sequence may similarly be the same nucleotide sequence as that in pgRNA. However, these nucleotide sequences in the evaluation nucleic acid for replication activity need to be converted into DNA. More specifically, U (uracil) in the nucleotide sequence should be replaced with T (thymine).

The evaluation nucleic acid for HBV replication activity can function by being incorporated in an appropriate gene expression system such as the evaluation vector for HBV replication activity according to the eighth aspect described below. As described above, HBV synthesizes pgRNA with RNA pol II of the host cell using the (−) strand of cccDNA as template. Subsequently, HBV resynthesizes the (−) strand DNA of HBV by the reverse transcription activity of HBV-pol using synthesized pgRNA as template. The evaluation nucleic acid for HBV replication activity according to the present invention also serves as template for the synthesis of a reporter pgRNA by RNA pol II when expressed in the host cell (FIG. 2B) and the intron in the reporter sequence is removed (spliced out) by pre-mRNA splicing upon the synthesis (FIG. 2B). Subsequently, DNA illustrated in FIG. 2C is synthesized by the reverse transcription activity of HBV-pol using the reporter pgRNA as template. As used herein, the DNA synthesized from this reporter pgRNA by reverse transcription is referred to as the “reporter minus strand DNA” or the “reporter (−) strand DNA”. This reporter (−) strand DNA corresponds to the (−) strand DNA of HBV. More specifically, the reporter (−) strand DNA would be generated only when the replication of HBV progresses and therefore the amount of synthesized reporter (−) strand DNA would reflect the amount of synthesized (−) strand DNA by the reverse transcription activity of HBV-Pol using pgRNA as template. Here, whereas the evaluation nucleic acid for HBV replication activity has intron in the reporter sequence, the reporter sequence of reporter (−) strand DNA does not have intron. Therefore, the evaluation nucleic acid for HBV replication activity that has served as template and the newly replicated reporter (−) strand DNA can be distinguished based on the presence or absence of intron. Therefore, the replication activity of HBV can be quantified by detecting the reporter sequence from which the intron has been removed in host cells. Specifically, the detection can be done using a primer pair for detecting reporter minus strand DNA described below.

### 8. Evaluation Vector for Hepatitis B Virus Replication Activity (Evaluation Vector for HBV Replication Activity)

### 8-1. Summary

The eighth aspect of the present invention is a vector that makes it possible to evaluate replication activity in hepatitis B virus (evaluation vector for HBV replication activity). The evaluation vector for HBV replication activity according to the present invention is composed of an expression vector comprising the evaluation nucleic acid for HBV replication activity according to the seventh aspect in a state that allows for the expression. This evaluation vector for HBV replication activity is a central expression vector in the evaluation system for HBV replication activity according to the ninth aspect described below and the replication activity of HBV can be quantified by detecting the reporter minus strand DNA generated by reverse transcription of the reporter pgRNA expressed from this expression vector.

### 8-2. Constitution

The evaluation vector for HBV replication activity according to this aspect is an expression vector and comprises a promoter and the evaluation nucleic acid for HBV replication activity according to the seventh aspect as essential components.

The “expression vector” refers to a vector comprising a gene or a gene fragment in a state that allows for the expression and comprising an expression unit that can control the expression of the gene or the like. As used herein, “a state that allows for the expression” refers to placing the gene to be expressed in the promoter-downstream region under the control of the promoter. As used herein, the “expression vector” refers to a vector comprising the evaluation nucleic acid for HBV replication activity according to the seventh aspect in a state that allows for the expression. Known vectors include plasmid vectors and viral vectors and any vector can be used. Usually, a plasmid vector, with which genetic recombination is easy, is sufficient. The expression vector may be a commercially available expression vector for mammalian cells. Examples thereof include the pCI vector or the pSI vector from Promega Corporation. Moreover, the expression vector may be a shuttle vector that can replicate both in mammalian cells and a bacterium such as Escherichia coli.

The promoter in the evaluation vector for HBV replication activity according to this aspect is a promoter capable of inducing gene expression in a host cell. Since the host cell which the evaluation vector for HBV replication activity is introduced into is in principle a mammalian cell, in particular a cell derived from human or chimpanzee, the promoter may be a promoter that is capable of expressing a downstream gene in such cells. Examples thereof include CMV promoter (CMV-IE promoter), SV40 early promoter, RSV promoter, EF1α promoter, and Ub promoter.

In the evaluation vector for HBV replication activity, the evaluation nucleic acid for HBV replication activity according to the seventh aspect is placed downstream of the promoter in a state that allows for the expression. The activation of the promoter induces the expression of the evaluation nucleic acid for replication activity.

The evaluation vector for HBV replication activity may comprise, in addition to the aforementioned essential components, an enhancer, a terminator, a multicloning site, a selection marker, and a replication origin.

The evaluation vector for HBV replication activity needs no replication origin for mammalian cells since transient expression of the vector is in principle sufficient in mammalian cells. However, when the evaluation vector for HBV replication activity is expressed as a shuttle vector in a bacterium such as Escherichia coli, a replication origin therefor is required. The replication origin may be a replication origin having a known sequence. For example, fl origin may be used as a replication origin for Escherichia coli.

### 9. Evaluation System for Hepatitis B Virus Replication Activity (Evaluation System for HBV Replication Activity)

### 9-1. Summary

The ninth aspect of the present invention is a system that makes is possible to evaluate replication activity in hepatitis B virus (evaluation system for HBV replication activity). The evaluation system for HBV replication activity in this aspect is an assay system that imitates the mechanism of HBV replication and composed of, in addition to the evaluation vector for HBV replication activity according to the eighth aspect, an expression vector comprising some auxiliary genes. The replication activity of HBV can be quantified and evaluated by introducing these expression vectors into host cells; culturing the host cells, and then detecting and quantifying the reporter minus strand DNA generated by reverse transcription of the reporter pgRNA derived from the evaluation vector for HBV replication activity according to the eighth aspect in the cells.

### 9-2. Constitution

The evaluation system for HBV replication activity comprises the evaluation vector for HBV replication activity according to the eighth aspect and expression vectors for the C and P genes of HBV as essential components and comprises an expression vector for the X gene of HBV and a primer pair for detecting reporter minus strand DNA as optional components.

The components: the evaluation vector for HBV replication activity, the HBc expression vector, the HBV-P expression vector, the HBx expression vector, and the primer pair for detecting reporter minus strand DNA will be described below.

### (1) Evaluation Vector for HBV Replication Activity

The constitution of the evaluation vector for HBV replication activity is as described in the eighth aspect. Therefore, specific descriptions are omitted here.

### (2) HBc Expression Vector

The “HBc expression vector” is an expression vector comprising the C gene in HBV in a state that allows for the expression. The “C gene” is an essential gene for HBV replication encoding the core protein HBc that constitutes the nucleocapsid of HBV. The C gene also consists of a nucleotide sequence that is different depending on the genotype. For example, HBc of HBV/A (herein, often referred to as “HBc/A”. Other genotypes are also referred to similarly.) consists of the amino acid sequence set forth in SEQ ID NO: 16, HBc/B consists of the amino acid sequence set forth in SEQ ID NO: 17, HBc/C consists of the amino acid sequence set forth in SEQ ID NO: 18, HBc/D consists of the amino acid sequence set forth in SEQ ID NO: 19, HBc/E consists of the amino acid sequence set forth in SEQ ID NO: 20, HBc/F consists of the amino acid sequence set forth in SEQ ID NO: 21, HBc/G consists of the amino acid sequence set forth in SEQ ID NO: 22, and HBc/H consists of the amino acid sequence set forth in SEQ ID NO: 23. A C gene is composed of a nucleotide sequence encoding one of the aforementioned HBcs. Examples thereof include the C gene of Genotype C consisting of the nucleotide sequence set forth in SEQ ID NO: 24 encoding HBc/C set forth in SEQ ID NO: 18 or a human codon-optimized sequence set forth in SEQ ID NO: 25 artificially designed based thereon.

The HBc expression vector may be an expression vector capable of expressing the contained C gene in host cells. The basic constitution may be in accordance with that of the aforementioned evaluation vector for HBV replication activity. The promoter used in the expression of the C gene is also in principle a promoter capable of inducing the expression of a downstream gene in mammalian cells, like the evaluation vector for HBV replication activity. Moreover, a commercially available expression vector for mammalian cells may be used. Examples thereof include the pCI vector or the pSI vector from Promega Corporation.

### (3) HBV-P Expression Vector

The “HBV-P expression vector” is an expression vector comprising the P gene in HBV in a state that allows for the expression. The “P gene” is an essential gene for HBV replication encoding the DNA polymerase HBV-Pol in HBV, as described above. As described above, 8 genotypes A to H are known for HBV-Pol and the amino acid sequences of respective genotypes are different as set forth in SEQ ID NOs: 1 to 8. The P gene contained in the HBV-P expression vector may be composed of a nucleotide sequence encoding any HBV-Pol. Examples thereof include the nucleotide sequence set forth in SEQ ID NO: 26 of the P gene encoding HBV/C-Pol or a human codon-optimized sequence set forth in SEQ ID NO: 27 artificially designed based thereon.

The HBV-P expression vector may be an expression vector capable of expressing the contained P gene in host cells. The basic constitution may be in accordance with that of the aforementioned evaluation vector for HBV replication activity. The promoter used in the expression of the P gene is also in principle a promoter capable of inducing the expression of a downstream gene in mammalian cells, like the evaluation vector for HBV replication activity. Moreover, a commercially available expression vector for mammalian cells may be used. Examples thereof include the pCI vector or the pSI vector from Promega Corporation.

### (4) HBx Expression Vector

The “HBx expression vector” is an expression vector comprising the X gene in HBV in a state that allows for the expression. The “X gene” is a gene encoding HBx, a protein that transactivates transcription of other genes in HBV. The X gene also consists of a nucleotide sequence that is different depending on the genotype. For example, HBx of HBV/A (herein, often referred to as “HBx/A”. Other genotypes are also referred to similarly.) consists of the amino acid sequence set forth in SEQ ID NO: 28, HBx/B consists of the amino acid sequence set forth in SEQ ID NO: 29, HBx/C consists of the amino acid sequence set forth in SEQ ID NO: 30, HBx/D consists of the amino acid sequence set forth in SEQ ID NO: 31, HBx/E consists of the amino acid sequence set forth in SEQ ID NO: 32, HBx/F consists of the amino acid sequence set forth in SEQ ID NO: 33, HBx/G consists of the amino acid sequence set forth in SEQ ID NO: 34, and HBx/H consists of the amino acid sequence set forth in SEQ ID NO: 35. An X gene is composed of a nucleotide sequence encoding one of the aforementioned HBxs. Examples thereof include the nucleotide sequence set forth in SEQ ID NO: 36 encoding HBx/C set forth in SEQ ID NO: 30 or a human codon-optimized sequence set forth in SEQ ID NO: 37 artificially designed based thereon. The HBx expression vector is not an essential component for the evaluation system for HBV replication activity, but it may be included in the evaluation system for HBV replication activity as needed, since the presence of HBx can promote the reverse transcription activity of HBc and HBV-Pol.

The HBx expression vector may be an expression vector capable of expressing the contained X gene in host cells. The basic constitution may be in accordance with that of the aforementioned evaluation vector for HBV replication activity. The promoter used in the expression of the X gene is also in principle a promoter capable of inducing the expression of a downstream gene in mammalian cells, like the evaluation vector for HBV replication activity. Moreover, a commercially available expression vector for mammalian cells may be used. Examples thereof include the pCI vector or the pSI vector from Promega Corporation.

The genes contained in the expression vectors: the evaluation vector for HBV replication activity, the HBc expression vector, the HBV-P expression vector, and the HBx expression vector may be respectively contained in different expression vectors or 2 or more of the genes may be contained in one expression vector; for example, the C gene and the P gene may be contained in one expression vector, the C gene and the X gene may be contained in one expression vector, the P gene and the X gene may be contained in one expression vector, and the C gene, the P gene, and the X gene may be contained in one expression vector. Alternatively, any one or more of the C gene, the P gene, and the X gene may be contained in the evaluation vector for HBV replication activity. Moreover, when 2 or more genes are expressed in one expression vector, the genes may be placed under control of the same promoter or may be placed under control of different promoters; for example, when the C gene and the P gene are contained in one expression vector, the C gene and the P gene may be placed under control of the same CMV promoter or the C gene and the P gene may be respectively placed under control of the CMV promoter and the SV40 promoter.

### (5) Primer Pair for Detecting Reporter Minus Strand DNA

The “primer pair for detecting reporter minus strand DNA” is constructed to make it possible to detect the presence or absence of intron in a reporter sequence contained in the aforementioned evaluation vector for HBV replication activity by nucleic acid amplification. The design of the primer pair is not particularly limited. For example, the primer pair may be designed so that only the reporter sequence in the reporter minus strand DNA is amplified.

For designing the primer pair so that only the reporter sequence in the reporter minus strand DNA, that is, the reporter sequence from which the intron has been removed is amplified, either of the forward primer or the reverse primer may be designed to extend across the exon junction in the reporter sequence. For this, designing the primer to extend across the exon junction with the 2 to 3 nucleotides in the elongation direction would conveniently reduce the background due to nonspecific amplification. For example, when designing the forward primer to extend across the exon junction, the forward primer may be designed so that only 2 to 3 nucleotides at the 3′ terminal side thereof match the 2 to 3 nucleotides at the 5′ terminal of the downstream exon.

The genotypes of the HBV-derived genes constituting the evaluation vector for HBV replication activity, the HBc expression vector, the HBV-P expression vector, and the HBx expression vector may be any combination of genotypes, but it is preferred to use the same genotype of genes. For example, if the evaluation nucleic acid for replication activity contained in the evaluation vector for HBV replication activity is Genotype C, then the C gene contained in the HBc expression vector, the P gene contained in the HBV-P expression vector, and the X gene contained in the HBx expression vector are preferably of Genotype C, the same genotype as the evaluation nucleic acid for replication activity, although they may be of different genotypes.

The evaluation system for HBV replication activity according to the present invention may be used as a kit. In such a case, the kit may comprise, in addition to the aforementioned essential components and optional components, a DNA extraction reagent, a reagent for nucleic acid amplification, a spin column, an instruction, and the like.

### 10. Method for Evaluating Hepatitis B Virus Replication Activity (Method for Evaluating HBV Replication Activity)

### 10-1. Summary

The tenth aspect of the present invention is a method for evaluating replication activity in hepatitis B virus (method for evaluating HBV replication activity). According to the method for evaluating HBV replication activity according to the present invention, HBV replication can be analyzed safely, inexpensively and rapidly at high-throughput, using various cells other than hepatocytes from mammals other than human, but without using infectious HBV virus.

### 10-2. Method

The method for evaluating HBV replication activity according to this aspect comprises an introduction step, a culturing step, an extraction step, and a detection step as essential steps. Each of the steps will be described below.

### (1) Introduction Step

The “introduction step” is a step of introducing the evaluation vector for HBV replication activity and the expression vectors in the evaluation system for HBV replication activity according to the ninth aspect into host cells. In this step, a cell line with which the evaluation of the HBV replication activity is possible is prepared.

Essential expression vectors to be introduced are the evaluation vector for HBV replication activity, the HBc expression vector, and the HBV-P expression vector and the HBx expression vector may be introduced as needed. The introduction ratio of the expression vectors is not limited, but among the C, P, and X genes, preferably the C gene is the highest, the P gene is at the second highest, and the X gene is the lowest. Examples of such a ratio include the ratio C:P:X=9:3:1. Moreover, the introduction ratio of the evaluation nucleic acid for HBV replication activity and other genes is also not limited, but it is preferred that the evaluation nucleic acid for HBV replication activity is the highest. Examples of such a ratio include the ratio the evaluation nucleic acid for HBV replication activity:C+P+X=1:1.

The host cells may be mammalian cells. Preferably, the host cells are cells derived from a human or a chimpanzee, which are HBV hosts. The type of cells of the origin is not limited. Cells derived from various organs and tissues other than hepatocytes, which are cells that are infected with HBV, may be used. Moreover, the host cells may be either a cell line or a primary culture cell line. Specific examples of the host cells include HeLa cells, HEK293 cells, NIH3T3 cells, and CHO cells.

The method of introducing expression vectors to the host cells is not particularly limited. A method of transfection (method of transformation) known in the art, as described in Green & Sambrook, 2012, Molecular Cloning: A Laboratory Manual Fourth Ed., Cold Spring Harbor Laboratory Press, Cold Spring Harbor, N.Y. or the like, may be used. The host cells may be transfected, for example, by using Lipofectin (PNAS, 1989, 86: 6077, PNAS, 1987, 84: 7413), electroporation, the calcium phosphate method (Virology, 1973, 52: 456-467), or the DEAE-Dextran method.

### (2) Culturing Step

The “culturing step” is a step of culturing host cells after the introduction. This step aims to produce the reporter minus strand DNA in the cells by expressing the proteins HBc, HBV-Pol, and HBx from the introduced expression vector and reverse-transcribing the reporter pgRNA derived from the evaluation nucleic acid for HBV replication activity.

The medium used in this step may be a medium known in the art and generally used in cell culturing. Usually, a standard medium for cell culturing is sufficient. The “standard medium for cell culturing”, as stated here, mainly refers to a versatile basal medium, which is used in culturing various types of cells derived from mammals. Specific examples thereof include Eagle MEM (Eagle Minimum Essential Medium), DMEM (Dulbecco's Modified Eagle Medium), Ham F10 (Ham's Nutrient Mixture F10) medium, Ham F12 (Ham's Nutrient Mixture F12) medium, M199 medium, High Performance Medium 199, RPMI-1640 (Roswell Park Memorial Institute-1640) medium, and DMEM/F12 (Dulbecco's Modified Eagle Medium/Ham's Nutrient Mixture F12) medium. When using DMEM/F12 medium, the mixed ratio thereof is not particularly limited. Preferably, DMEM and F12 may be mixed in a range of from 6:4 to 4:6 in terms of weight concentration ratio of components.

The specific compositions of the standard media for cell culturing are known in the art and such a medium may therefore be prepared based on its composition described in appropriate documents (for example, Kaech S. and Banker G., 2006, Nat. Protoc., 1 (5): 2406-2415). Alternatively, media commercially available from manufacturers such as Thermo Fisher Scientifics Inc. and Wako Pure Chemical Industries, Ltd. may be used.

The method of culturing cells in this step may be a method of culturing known in the art according to the type and origin of the host cells. Usually, inoculating a medium for cell culturing with the cells and then culturing the resulting culture at 5% CO₂ and 37° C. is sufficient.

The culture period slightly varies depending on the type and origin of the host cells, but may be usually 12 hours to 72 hours and preferably 24 hours to 48 hours to achieve the aforementioned aim of this step.

### (3) Extraction Step

The “extraction step” is a step of extracting DNA from the host cells after the culturing step. This step aims to extract from the host cells DNA comprising reporter minus strand DNA synthesized by reverse transcription of the reporter pgRNA derived from the evaluation nucleic acid for HBV replication activity.

The method of extracting DNA may be a method known in the art for extracting DNA from cultured cells. For example, the extraction may be conducted by a method described in Green & Sambrook (supra). Moreover, kits for nucleic acid extraction from mammalian cells commercially available from manufacturers may be used.

### (4) Detection Step

The “detection step” is a step of detecting the reporter sequence from which the intron has been removed in the reporter minus strand DNA obtained in the extraction step. The HBV replication activity can be quantitatively analyzed by detecting and quantifying the reporter sequence from which the intron has been removed in this step.

The DNA obtained in the extraction step is a mixture of genomic DNA of the host cells, the genes derived from HBV and the evaluation nucleic acid for HBV replication activity introduced in the introduction step, the reporter minus strand DNA synthesized by reverse transcription of the reporter pgRNA derived from the evaluation nucleic acid for HBV replication activity, and the like. In this step, only the reporter sequence from which the intron has been removed in the reporter minus strand DNA is specifically detected among them.

The method of detection is not particularly limited, as long as it is a method that makes it possible to detect the reporter sequence from which the intron has been removed in the reporter minus strand DNA. Examples thereof include methods of nucleic acid amplification involving amplifying the reporter sequence using a primer pair and methods of hybridization involving detecting the junction of the exons using a probe.

### (4-1) Method of Nucleic Acid Amplification

The “methods of nucleic acid amplification” refer to methods involving amplifying a particular region of target nucleic acid by a nucleic acid polymerase using a primer pair. Examples thereof include PCR, NASBA, ICAN, LAMP® (including RT-LAMP). A preferable method of nucleic acid amplification is PCR. In particular, when the amount of the reporter minus strand DNA present in the DNA obtained in the extraction step is measured, it is desirable to use a quantitative method of nucleic acid amplification such as real-time PCR.

The primer pair may be the aforementioned primer pair for detecting reporter minus strand DNA. The primer pair that amplifies only the reporter sequence in the reporter minus strand DNA is preferred.

The reaction conditions for the real-time PCR may be those in a method known in the artand may be determined with referring to a method described in Green & Sambrook (supra). In general, since conditions change depending on the nucleotide length of the nucleic acid fragment to be amplified and the amount of the nucleic acid for template, the nucleotide length and Tm value of the primers to be used, the optimal reaction temperature and optimal pH for the nucleic acid polymerase, and the like to be used, the PCR conditions may be determined as appropriate based on the conditions for known PCR method and depending on these conditions. As an example, approximately 15 to 40 cycles of denaturation at 94 to 95° C. for 5 seconds to 5 minutes, annealing at 50 to 70° C. for 10 seconds to 1 minute, and extension at 68 to 72° C. for 30 seconds to 3 minutes and subsequent final extension at 68 to 72° C. for 30 seconds to 10 minutes may usually be performed. When a commercially available kit is used, the reactions may be performed according to a protocol attached to the kit in principle.

The nucleic acid polymerase used in real-time PCR is a DNA polymerase, in particular, a thermostable DNA polymerase. Various types of such nucleic acid polymerases are commercially available and may be used.

### (4-2) Method of Hybridization

The “methods of hybridization” are methods involving using as a probe a nucleic acid fragment having a sequence complementary to all or part of the nucleotide sequence of a target nucleic acid to be detected and detecting and quantifying the target nucleic acid or fragment thereof based on the base-pairing between the nucleic acid and the probe. Although some methods of hybridization that are different in means for detection are known, for example, Southern hybridization, RNA microarray, surface plasmon resonance, or quartz crystal microbalance is preferred, since the reporter minus strand DNA of the target nucleic acid is DNA.

The “southern hybridization” is the most common method for analyzing DNA. DNA extracted from a sample is separated by electrophoresis on agarose gel, polyacrylamide gel, or the like under denaturing conditions and transferred (blotted) onto a filter. Subsequently, a probe containing a sequence complementary to a target DNA-specific nucleotide sequence is used to detect the target DNA. The target DNA can also be quantified by labelling the probe with an appropriate marker such as a fluorescent dye or a radioisotope and using a measuring apparatus, for example, chemilumi (chemiluminescence) imaging and analyzing apparatus (for example, Light Capture, ATTO Technology, Inc.), Scintillation counter, imaging analyzer (for example, FUJIFILM Corporation: BAS series), or the like. Southern hybridization is a well-known technique in the art and described, for example, in Green, M. R. and Sambrook, J. (supra).

The probe may be a probe that hybridizes only to the reporter sequence in the reporter minus strand DNA, a combination of a probe that specifically hybridizes to the intron and a probe that hybridizes to only one exon or the whole reporter sequence, or the like, but the former probe is preferred. An example of the probe that hybridizes only to the reporter sequence in the reporter minus strand DNA may be a probe having a nucleotide sequence complementary to the exon junction in the reporter sequence after mRNA-splicing and designed not to hybridize only to one exon under stringent conditions.

### EXAMPLES

### Example 1: Establishment of Evaluation Method for HBV Replication Activity

### (Purpose)

A safe, inexpensive and quick evaluation method for HBV replication activity that can visualize and quantify replication of HBV genome in a short time using general cells without using infectious HBV is constructed.

### (Method)

(1) Preparation of evaluation vector for HBV replication activity (pBB-intron)

In HBV pgRNA of genotype C, the sequence between 5′-terminal 99 nucleotides (corresponding to positions 32-130 of HBV pgRNA) containing a first DR1 sequence and a first ε sequence and 3′-terminal 349 nucleotides (corresponding to positions 3013-3215 and positions 1-146 of HBV pgRNA) containing DR2 sequence, a second DR1 sequence and a second ε sequence; in other words, first c/DR2 sequence (corresponding to positions 131-3012 of HBV pgRNA), was replaced with a 320-nucleotide reporter sequence containing an intron to prepare a nucleic acid for evaluating an HBV replication activity, represented by SEQ ID NO: 15 and described in the 7th aspect of the present invention (FIG. 2A). The reporter sequence is derived from nucleotide sequences of 483-site to 453-site (31 base pairs) of pCRE-MetLuc 2 vector (Clontech), 857-site to 989-site (133 base pairs) of pCI vector (Promega), and 452-site to 337-site (116 base pairs) of pCRE-MetLuc2 vector.

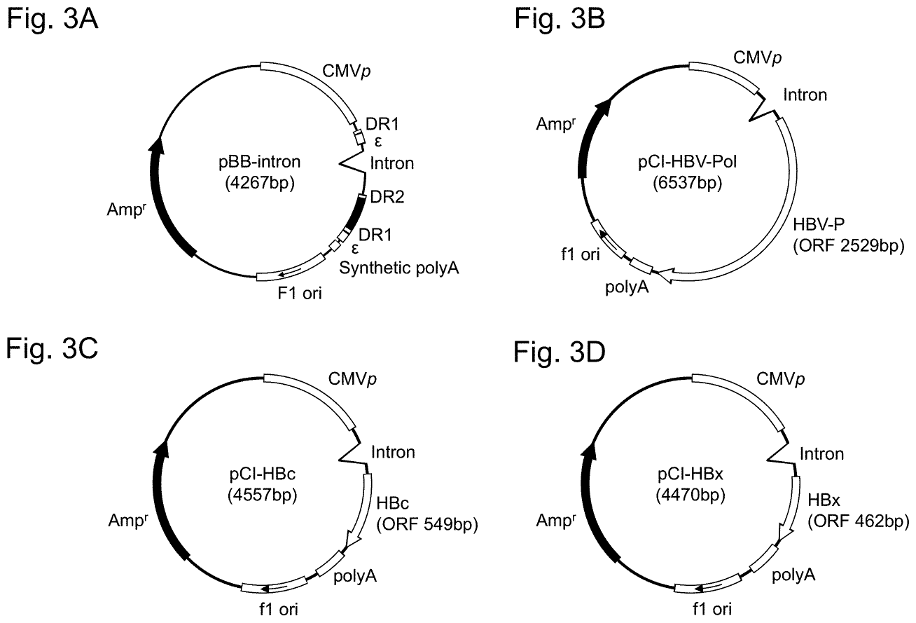

A CMV promoter (742 base pairs) and TATA-box (46 base pairs) were inserted upstream of the evaluation nucleic acid for HBV replication activity prepared, and synthetic polyA signal sequence (49 base pairs) was inserted downstream thereof. The resultant entire sequence was inserted in the backbone (containing only a phage fl origin, an ampicillin resistance gene and a replication origin) of pCI vector to prepare pBB-intron, which is an evaluation vector for HBV replication activity, described in the eighth aspect of the present invention (FIG. 3A).

When the evaluation vector for HBV replication activity is introduced in a host cell, mRNA is synthesized by RNA-pol II of the host cell. The mRNA is synthesized as pre-mRNA, and thereafter, the intron in the reporter sequence is immediately removed by pre-mRNA splicing within the host cell. The mature mRNA from which the intron has been spliced out corresponds to reporter pgRNA (FIG. 2B). The reporter pgRNA is reverse-transcribed by the function of HBV-Pol, HBc and HBx expressed by an HBV-P expression vector (described later), an HBc expression vector and an HBx expression vector, respectively to synthesize a reporter-minus strand DNA containing a reporter sequence from which the intron has been removed, which differs from the reporter sequence of an original evaluation vector for HBV replication activity (FIG. 2C).

(2) Preparation of HBV-P expression vector (pCI—HBV-P)

The sequence represented by SEQ ID NO: 27, which was artificially designed based on P gene of HBV represented by SEQ ID NO: 26, was inserted in pCI vector (Promega) under control of CMV promoter to prepare an HBV-P expression vector, i.e., pCI-HBV-P, described in the eighth aspect of the present invention (FIG. 3B).

(3) Preparation of HBc expression vector (pCI-HBc)

The sequence represented by SEQ ID NO: 25, which was artificially designed based on C gene of HBV represented by SEQ ID NO: 24, was inserted in pCI vector (Promega) under control of CMV promoter to prepare an HBV-C expression vector, i.e., pCI-HBc, described in the eighth aspect of the present invention (FIG. 3C).

(4) Preparation of HBx expression vector (pCI-HBx)

The sequence represented by SEQ ID NO: 37, which was artificially designed based on X gene of HBV represented by SEQ ID NO: 36, was inserted in pCI vector (Promega) under control of CMV promoter to prepare an HBx expression vector, i.e., pCI-HBx, described in the eighth aspect of the present invention (FIG. 3D).

(5) Method for evaluating HBV replication activity

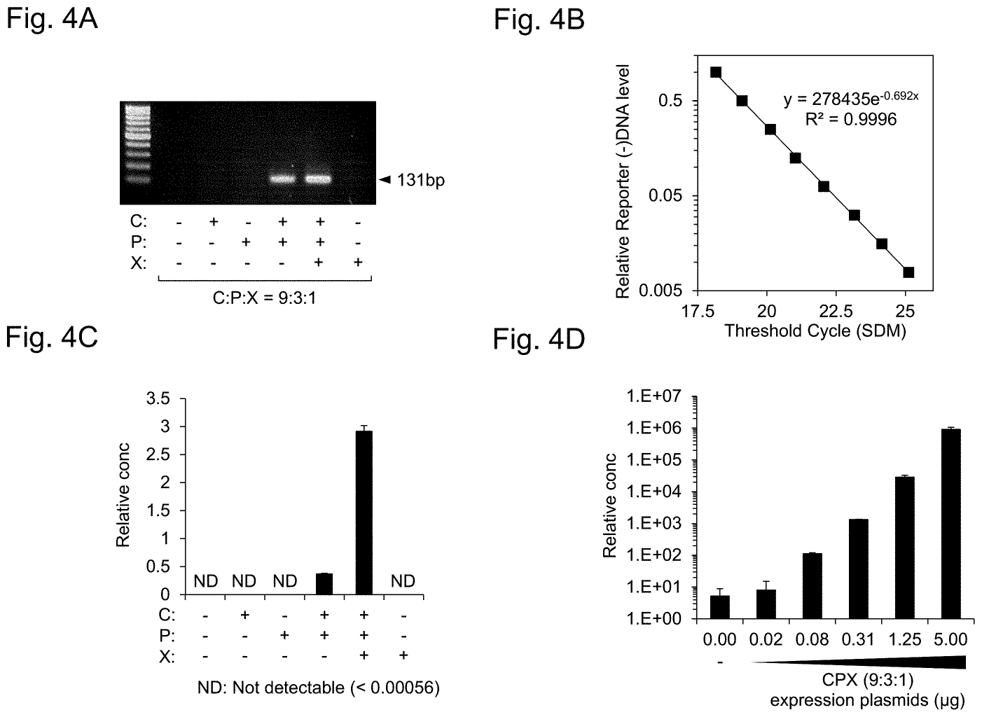

To HeLa cells, the evaluation vector for HBV replication activity, HBV-P expression vector, HBc expression vector and HBx expression vector were introduced in various combinations (Introduction step). Combination patterns are as described in FIG. 4A. Transfection of the HeLa cells was carried out by electroporation. DNA (10 μg) was introduced into about 1×10⁶ HeLa cells in the conditions of 125 V/2.5 ms plus length by an electroporator, Nepa 21 (Nepa Gene Co., Ltd.). The ratio of HBc expression vector:HBV-P expression vector:HBx expression vector was set to be 9:3:1. If there was an expression vector not to be introduced depending on the combination, the same ratio of an empty vector (pCI) as that of the expression vector not to be introduced was introduced. The ratio of the evaluation vector for HBV replication activity and the total of other expression vectors was set to be 1: 1 (5 μg: 5 μg). If there was an expression vector not to be introduced, pCI was introduced at the ratio corresponding to that of the expression vector.

Furthermore, to confirm whether the amount of reporter-minus strand DNA obtained by reverse transcription reflects the functions of HBc, HBV-Pol, and HBx in a cell, the following analysis was made:

When the ratio of evaluation vector for HBV replication activity:the HBc/HBV-P/HBx expression vectors was changed stepwise from 1:0 (5 μg:pCI 5 μg), 1:0.004 (5 μg:0.02 μg), 1:0.016 (5 μg:0.08 μg), 1:0.062 (5 μg:0.31 μg), 1:0.25 (5 μg:1.25 μg) to 1:1 (5 μg:5 μg) without changing the ratio of HBc:HBV-P:HBx expression vectors (HBc:HBV-P:HBx=9:3:1), the effect on the reverse transcription reaction was analyzed based on the reporter-minus strand DNA amount.

After transfection, the HeLa cells were cultured in DMEM supplemented with 2 mL of 10% FBS in the presence of 5% CO₂ at 37° C. for 48 hours (Culturing step).

After culture, DNA was extracted from the HeLa cells by means of DNeasy Mini (Qiagen). As the primer, a primer pair of a forward primer consisting of the nucleotide sequence represented by SEQ ID NO: 38 and a reverse primer consisting of the nucleotide sequence represented by SEQ ID NO: 39, was used. The forward primer has the two nucleotides at the 3′ terminal, which match the two nucleotides at the 5′ terminal of the exon present on the downstream side; however, fail to match two nucleotides at the 5′ terminal of the intron. Accordingly, the forward primer functions as a primer only in the case where reporter-minus strand DNA having a reporter sequence from which the intron was removed is present, and amplification can be made by using the reporter-minus strand DNA as a template to provide a DNA fragment of 131 nucleotides.

DNA derived from cells to which expression vectors were introduced in the combinations described in FIG. 4A were subjected to PCR, and thereafter, PCR products were separated by 2% agarose-gel electrophoresis and stained with ethidium bromide.

In order to prepare a calibration curve, a template DNA was prepared by serially diluting DNA derived from HeLa cells, in which expression vectors were introduced so as to satisfy the ratio of HBc:HBV-P:HBx expression vectors of 9:3:1, and the ratio of the evaluation vector for HBV replication activity: HBc/HBV-P/HBx expression vectors of 1:1, and then, subjecting the template DNA to real time PCR.

### (Results)

The results of Example 1 are shown in FIG. 4.

A shows the results of 2% agarose-gel electrophoresis of PCR products derived from cells to which expression vectors were introduced in various combinations and stained with ethidium bromide. Reference symbol C represents an HBc expression vector containing C gene; P represents an HBV-P expression vector containing P gene; and X represents an HBx expression vector containing X gene. Symbol + indicates introduction in HeLa cells; symbol - indicates no introduction. The 131-bp PCR product derived from reporter-minus strand DNA was observed only when HBc expression vector and HBV-P expression vector were introduced. From this, it was found that HBc and HBV-Pol are essential for reverse transcription of pgRNA. It was also confirmed that HBx is not essential for reverse transcription of pgRNA; however, it remarkably promotes the reverse transcription by HBc and HBV-Pol.

B shows a calibration curve prepared based on the results of real time PCR of serially diluted DNAs. As shown in the figure, an accurate calibration curve (R²=0.9996) was obtained. It was demonstrated that the amount of reporter-minus strand DNA obtained by reverse transcription can be quantified by real time PCR.

C shows quantitative analysis results based on the real time PCR and the calibration curve of B; more specifically the results obtained by amplifying DNA samples used in A in the above by real time PCR, and thereafter, quantitatively analyzing the amplified product based on the calibration curve (shown in B) in the above. Almost the same results as in the analysis results by agarose-gel electrophoresis (shown in A) were obtained.

D is a graph showing the correlation between the expression level of HBV protein (HBc, HBV-P, HBx) and the amount of HBV DNA replication, in other words, reporter-minus strand DNA. The reverse transcription of pgRNA exponentially increased in accordance with the expression level of HBV protein. From the results, it was found that, according to the evaluation method for HBV replication activity using the evaluation system for HBV replication activity of the present invention, the function of HBV protein in a host cell can be reflected and the activity of reverse transcription, i.e., replication activity, can be measured and quantified.

### Example 2: Examination of Evaluation Method for HBV Replication Activity Using Entecavir

### (Purpose)

Using the evaluation system for HBV replication activity of the present invention, the effect of an existing anti-HBV drug, Entecavir, was studied.

### (Method)

In accordance with the evaluation method for HBV replication activity described in Example 1, an evaluation vector for HBV replication activity, HBV-P expression vector, HBc expression vector and HBx expression vector were introduced in HeLa cells. The introduction ratio of HBc:HBV-P:HBx expression vectors was set to be 9:3:1 and the introduction ratio of the evaluation vector for HBV replication activity and HBc+HBV-p+HBx expression vector was set to be 1:1 (5 μg:5 μg). After the introduction step, DMEM supplemented with 2 mL of 10% FBS was added to the HeLa cells, and then, the mixture was dispensed in wells of a 96 well-plate at 0.1 mL/well. To individual mediums, Entecavir was added at 0.04 μM/well, 0.08 μM/well, 0.16 μM/well, 0.31 μM/well, 0.63 μM/well, 1.25 μM/well, 2.50 μM/well, and 5.00 μM/well, and culture was carried out in the presence of 5% CO₂ at 37° C. for 24 hours. Entecavir is a powerful nucleotide/nucleoside analog formulation and known to act on HBV-Pol to inhibit reverse transcription (Billich A., 2001, Curr Opin Investig Drugs., 2(5):617-21).

After culture, 2% NP-40/Proteinase K was added in each well and DNA was extracted from the HeLa cells and subjected to real time PCR in accordance with the method of Example 1.

### (Results)

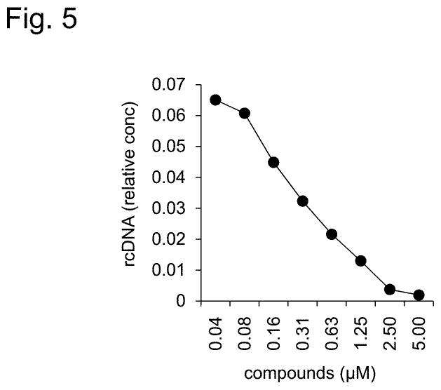

After amplification by Real time PCR, reporter-minus strand DNA was quantitatively analyzed based on the calibration curve prepared in Example 1 (FIG. 4B). The results are shown in FIG. 5. The amount of the reporter-minus strand DNA decreased in an Entecavir-concentration depending manner. The result suggests that replication of HBV DNA is inhibited in an Entecavir-concentration depending manner. Thus, it was demonstrated that an HBV replication process can be quantified in 24 hours, which is extremely short compared to conventional methods, by using the evaluation system for HBV replication activity of the present invention, without either using infectious HBV particles or producing HBV.

### Example 3: Search for Natural Compound Selectively Inhibiting Replication of HBV and Derived from Filamentous Fungus

### (Purpose)

A novel anti-HBV compound inhibiting replication of HBV is searched by using the evaluation system for HBV replication activity of the present invention.

### (Method)

(1) Search for natural compound having anti-HBV activity

Using the evaluation system for HBV replication of the present invention, a natural compound having an anti-HBV activity was searched from a filamentous fungus culture extract library (ExMyco: HyphaGenesis Inc.).

To HeLa cells, an evaluation vector for HBV replication activity, and expression vectors of HBV-P, HVc and HBx were introduced in the ratio described in the evaluation method for HBV replication activity described in Example 2. Thereafter, the HeLa cells were cultured in a 96-well plate containing a culture solution supplemented with a 0.25% n-butanol extract of a filamentous fungus culture, under 5% CO₂ atmosphere at 37° C. Twenty-four hours later, WST-8 (Nacalai) was added to the culture solution, which was further subjected to culture for 2 hours. The absorbance of the culture solution was measured at 450 nm/650 nm to evaluate the survival rates of the cells.

After the culture solution was removed, DNA in the cell cytoplasm was recovered by a treatment with 50 μl of Buffer A (10 mM HEPES, 1.5 mM MgCl₂, 10 mM KCl, 0.5 mM DTT, 0.05% NP40, pH7.9) and subjected to real time PCR performed in accordance with the method of Example 1. As the result of primary screening of 800 extracts (200 strains, 4 culture solutions), 162 extracts inhibiting 25% or more of HBV DNA replication (reverse transcription) and having a cytotoxic activity of 80% or less were selected as a primary hit.

Subsequently, 162 extracts primarily hit were serially diluted and added to an evaluation system. As a result of comparison and examination of anti-HBV activity and cytotoxic activity, a filamentous fungus strain (FA233 strain) was obtained, which produces an extract inhibiting replication of HBV DNA in a concentration-dependent manner and exhibiting no cytotoxic activity, when any type of four mediums (GP medium, MV8 medium, KGC medium, SC medium) was used for culturing. The FA233 strain was further cultured in the KGC medium in a large scale and a compound having an anti-HBV activity was isolated by silica gel chromatography and HPLC. Thereafter, the compound was serially diluted and re-studied for anti-HBV activity and cytotoxic activity.

### (Results)

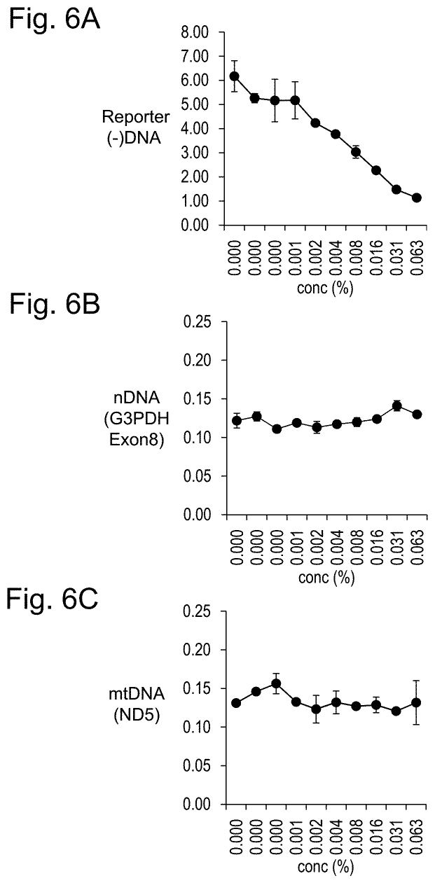

The results are shown in FIG. 6. The compound inhibited replication of HBV DNA in a concentration-dependent manner (FIG. 6A). However, the amounts of genomic DNA (G3PDH exon 8) and mitochondrial DNA (ND5) were not affected (FIG. 6B, C). Then, the compound was selected as an HBV DNA replication inhibitor.

### Example 4: Identification of HBV DNA Replication Inhibitor

### (Purpose)

The chemical structure of the HBV DNA replication inhibitor obtained in Example 3 is identified.

### (Method)

NMR samples (about 0.4 mL) of 10 mg/CDCl₃ 35 mm height was prepared; and subsequently, measured in accordance with various NMR (H-1, C-13, DEPT, COSY, HSQC, HMBC and NOESY) spectroscopies by JEOL ECA 500 (JEOL RESONANCE).

### (Results)

The NMR measurement results, values described in a reference literature, and comparison results between them are shown in Table 1. Reference symbol “lit” in Table stands for a reference literature (Agatsuma T., et al. 1993, Chem. Pharm. Bull., 41: 373-375).

TABLE 1

| | C-13 in CDCl3 | | | H-1 in CDCl3 | | |
|---|---|---|---|---|---|---|
| assignments | obs | lit* | Δδ | obs | lit* | Δδ |
| 10′-Me | 21.0 | 21.1 | −0.1 | 1.44 | 1.41 | 0.03 |
| 3′ | 34.5 | 34.6 | −0.1 | 1.12 | 1.10 | 0.02 |
| | | | | 2.00 | 1.96 | 0.04 |
| 9′ | 36.9 | 37.0 | −0.1 | 2.58 | 2.56 | 0.02 |
| | | | | 3.12 | 3.09 | 0.03 |
| 4-OMe | 55.5 | 55.5 | 0.0 | 3.80 | 3.77 | 0.03 |
| 1′ | 57.9 | 56.0 | 1.9 | 4.41 | 4.37 | 0.04 |
| 2′ | 62.5 | 62.6 | −0.1 | 2.90 | 2.88 | 0.02 |
| 4′ | 70.7 | 70.7 | 0.0 | 4.01 | 3.99 | 0.02 |
| 10′ | 73.2 | 73.2 | 0.0 | 5.50 | 5.49 | 0.01 |
| 5′ | 80.9 | 81.0 | −0.1 | 4.61 | 4.58 | 0.03 |
| 3 | 101.0 | 101.1 | −0.1 | 6.40 | 6.39 | 0.01 |
| 5 | 103.6 | 103.6 | 0.0 | 6.41 | 6.38 | 0.03 |
| 1 | 103.9 | 104.0 | −0.1 | | | |
| 7′ | 126.3 | 126.4 | −0.1 | 6.35 | 6.37 | −0.02 |
| 6 | 142.2 | 142.3 | −0.1 | | | |
| 8′ | 145.4 | 145.4 | 0.0 | 6.20 | 6.17 | 0.03 |
| 4 | 165.1 | 165.2 | −0.1 | | | |
| 2 | 166.2 | 166.2 | 0.0 | | | |
| COO | 171.1 | 171.2 | −0.1 | | | |
| 6′ | 199.5 | 199.5 | 0.0 | | | |
| 2-OH | | | | 12.06 | | |

When the compound, from which the signals of various NMR measurement data come out, was analyzed, the signals matched with those from Hypothemycin (0.1 or less in the case of C-13, 0.04 or less in the case of H-1) described in the aforementioned literature. Accordingly, it was concluded that the compound obtained in Example 3 is Hypothemycin represented by Formula (1).

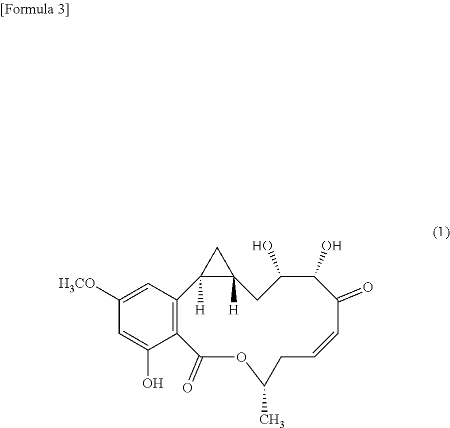 [SMILES: COc1cc(O)c2c(c1)[C@H]1C[C@@H]1C[C@H](O)[C@H](O)C(=O)/C=C\C[C@H](C)OC2=O]

### Example 5: Examination of HBV Replication-Inhibiting Effect by MAPK Kinase Inhibitor

### (Purpose)

Hypothemycin identified as an HBV DNA replication inhibitor in Example 4 is known as an inhibitor of MEK, which plays an important role in the classical MAPK pathway, more specifically, MAPK/ERK kinase (MAPKK) (Fukazawa H, et al., 2010, Biol Pharm Bull., 33 (2):168-73). However, it has been totally unknown that the MAPKK inhibitor has a medicinal effect as an anti-HBV drug.

Then, the inhibitory activity on HBV DNA replication by Hypothemycin will be re-studied; at the same time, whether other known MAPKK inhibitors have an HBV replication inhibitory activity similarly to Hypothemycin will be studied by use of the evaluation system for HBV replication activity of the present invention.

### (Method)

An evaluation vector for HBV replication activity, and HBV-P, HBc and HBx expression vectors were introduced in HeLa cells in the ratio used in the evaluation method for HBV replication activity described in Example 2. An MAPKK inhibitor (5 μM) was added to the HeLa cells, which were cultured under a 5% CO₂ atmosphere at 37° C. As the MAPKK inhibitor, four types of inhibitors: Hypothemycin, PD98059, PD184352 and

Trametinib, were used. A sample not containing an MAPKK inhibitor was used as a negative control and Entecavir (5 μM), which is not a MAPKK inhibitor but known as an anti-HBV drug, was used as a positive control for HBV replication inhibitory activity. The control samples were cultured in the same condition. Twenty-four hours later, DNA was extracted from the culture solution in accordance with the method described in Example 1 and subjected to real time PCR.

### (Results)

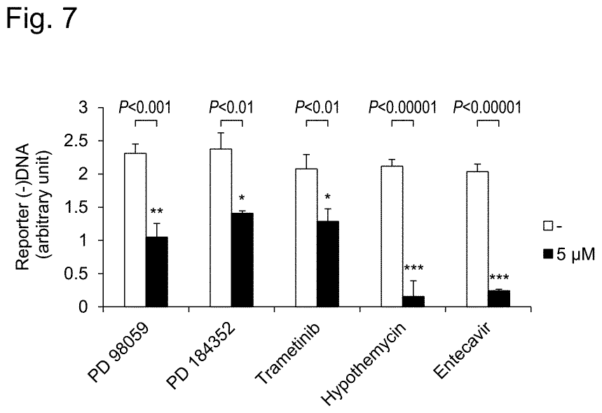

The results are shown in FIG. 7. The four types of MAPKK inhibitors examined all showed significant HBV DNA replication inhibitory activity at a concentration of 5 μM. From the result, a new finding that the MAPKK inhibitors are effective as an anti-HBV drug was obtained.

### Example 6: Action and Effect of MAPK Kinase on HBV DNA Replication

### (Purpose)

From the results of Example 5, it was found that the MAPKK inhibitors have an HBV DNA replication inhibitory activity. The result strongly suggests that the MAPK kinase activity plays an important role in HBV DNA replication. Then, the effect of a MAPK kinase on HBV DNA replication will be studied.

### (Method)

Forced expression of various human MEK1 in cells was performed to study the effect of MAPK kinase on HBV DNA replication. Specifically, the evaluation vector for HBV replication activity, and expression vectors of HBV-P, HBc and HBx were introduced in HeLa cells in the ratio used in the evaluation method for HBV replication activity described in Example 2; at the same time, various human MEK1 expression vectors, i.e., a wild-type (wild type: referred to as “Wt”), constitutively active (S²¹⁸E/S²²²E: referred to as “2E” S²¹⁸D/S²²²D: referred to as “2D”) or a dominant negative mutant (K⁹⁷A/S²²²A: referred to as “2A”), was separately introduced. After introduction of the various expression vectors, HeLa cells were cultured under a 5% CO₂ atmosphere at 37° C. Twenty-four hours after culture, DNA was extracted from each culture solution in accordance with the method described in Example 1 and subjected to real time PCR. The amounts of HBV DNA and genomic DNA by expression of MAPK kinase (MEK) and the effect of MEK1 expression on the amount of mitochondrial DNA were studied.

### (Results)

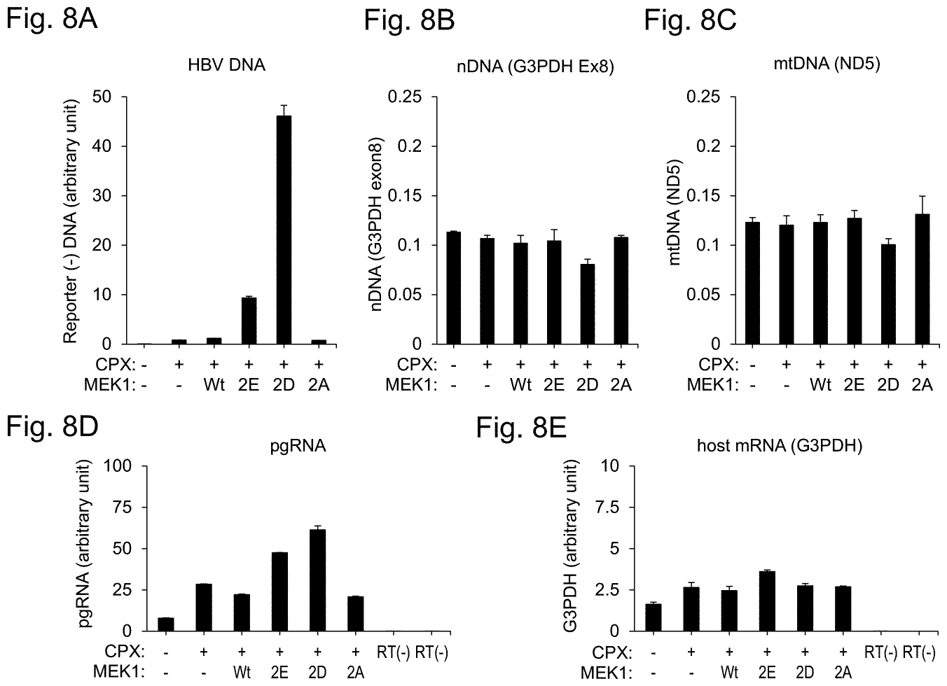

The results are shown in FIG. 8. It was found that HBV replication is promoted nearly 10-fold in the cells (2D, 2E) undergoing forced expression of constitutively active human MEK1 (FIG. 8A); in particular, promoted as much as 45-fold or more by the constitutively active form of 2D. In contrast, forced expression of human MEK did not affect the amounts of genomic DNA (FIG. 8B) and mitochondrial DNA (FIG. 8C). In addition, transcription of reporter pgRNA and host mRNA (G3PDH) were not affected.

From the results, it was confirmed that the MEK activity in host cells is important for replication of HBV DNA.

### Example 7: Phosphorylation of HBV Protein by MAPK Kinase

### (Purpose)

In order to elucidate the mechanism underlying promotion of HBV DNA replication by MAPK kinase, the stage of HBV DNA replication in which MAPK kinase has an effect is studied.

### (Method)

MAPK kinase, i.e., MEK, is a phosphorylation enzyme playing an important role in a classical MAPK signal transduction pathway. In the MAPK family (ERK, JNK1 and p38) serving as a substrate of MEK, a conservative sequence consisting of three amino acids, called as a “TxY motif” is present. When residue T and residue Y within the motif are phosphorylated by MEK, the MAPK family is activated (Morrison D. K., 2012, MAP kinase pathways. Cold Spring Harb Perspect Biol., 4 (11)). Accordingly, it was speculated that any of HBV proteins involved in replication of HBV DNA may be phosphorylated by MEK, also in HBV.

Amino acid sequences of HBV proteins (HBc, HBV-Pol, and HBx) involved in replication of HBV DNA were analyzed. As a result, it was found that a TxY motif is present at positions 120-122 in Terminal protein region of the HBV-Pol, and at positions 284-286 in the spacer region thereof (FIG. 9A). The TxY motif (¹²⁰TKY¹²²) in Terminal protein region was conserved in all genotypes A to F studied; whereas the TxY motif (²⁸⁴TAY²⁸⁶) in the spacer region was observed only in genotype C (HBV/C) (FIG. 9B).

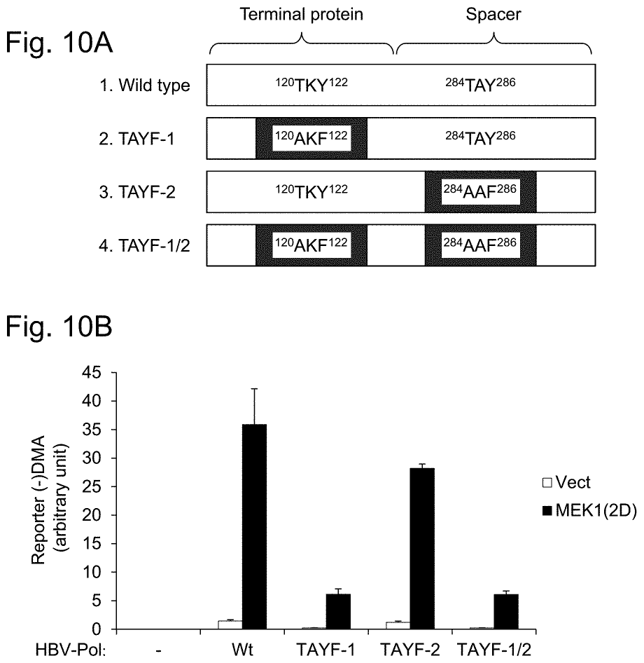

Then, in HBV-Pol of HBV/C, T in the TxY motifs was replaced with A (alanine) and Y was replaced with F (phenylalanine) to prepare mutant P genes encoding three mutant HBV/C-Pols (TAYF-1, TAYF-2, and TAYF-1/2) (FIG. 10A).

“TAYF-1” is mutant HBV/C-Pol containing ¹²⁰AKF¹²² in place of ¹²⁰TKY¹²² in Terminal protein region; “TAYF-2” is mutant HBV/C-Pol containing ²⁸⁴AAF²⁸⁶ in place of ²⁸⁴TAY²⁸⁶ in the spacer region; and “TAYF-1/2” is mutant HBV/C-Pol containing ¹²⁰AKF¹²² and ²⁸⁴AAF²⁸⁶ in place of ¹²⁰TKY¹²² in Terminal protein region and ²⁸⁴TAY²⁸⁶ in the spacer region, respectively. Expression vectors containing genes encoding mutant HBV/C-Pols of TAYF-1, TAYF-2, and TAYF-1/2 were designated as HBV/C-P-TAYF-1, HBV/C-P-TAYF-2 and HBV/C-P-TAYF-1/2, respectively.

HBV replication was evaluated in the same manner as in the evaluation method for HBV replication activity described in Example 1. The evaluation vector for HBV replication activity and wild type HBV/C-P (Wt) or mutant HBV-P, HBc and HBx expression vectors were introduced in the same ratio as in Example 2; at the same time, 2D expression vector (5 μM) for forced expression of constitutively active human MEK1 was introduced. Culture was carried out under a 5% CO₂ atmosphere at 37° C. Twenty-four hours after culture, DNA was extracted from each culture solution in accordance with the method described in Example 1 and subjected to real time PCR to study HBV replication activity.

### (Results)

The results are shown in FIG. 10B. In TAYF-1 and TAYF-1/2 where ¹²⁰TKY¹²² in Terminal protein region was mutated, it was found that the promoting activity of HBV replication is remarkably suppressed compared to the activity by forced expression of constitutively active MEK1 when a wild type (Wt) HBV/C-P was introduced. In contrast, TAYF-2 where a mutation of ²⁸⁴AAF²⁸⁶ alone was introduced in the spacer region, such suppression was not observed. From these results, it was suggested that HBV-Pol is activated by phosphorylation of ¹²⁰TKY¹²² positioned in Terminal protein region by MAPK kinase of a host cell.

From the results of Examples 6 and 7, it was suggested that a MAPKK inhibitor including Hypothemycin inhibits phosphorylation of its substrate HBV-Pol, via inhibition of activity of MAPKK, thereby suppressing activation of HBV-Pol to inhibit replication of HBV DNA.

### Example 8: Synergetic Effect of Known Anti-HBV Drug and MAPKK Inhibitor on HBV DNA Replication Inhibition

### (Purpose)

HBV DNA replication-inhibiting effect of a combined use of a known anti-HBV drug and a MAPKK inhibitor is studied.

### (Method)

As the known anti-HBV drug, Entecavir, which is known as a reverse transcription inhibitor in HBV replication, was used. As the MAPKK inhibitor, Hypothemycin was used.

The method for evaluating HBV replication activity of the present invention was in principle carried out in accordance with the method described in Example 1. The introduction ratio of individual expression vectors was in accordance with the ratio described in Example 2. To the cells having expression vectors introduced therein, DMEM supplemented with a 10% FBS together with all combinations (4×4=16 combinations) of Entecavir and Hypothemycin of 0 μM, 1.25 μM, 2.50 μM, and 5.00 μM were added, and thereafter, the cells were cultured in the presence of 5% CO₂ at 37° C.

Twenty-four hours later, DNA was extracted from the culture solution in accordance with the method described in Example 1 and subjected to real time PCR.

### (Results)

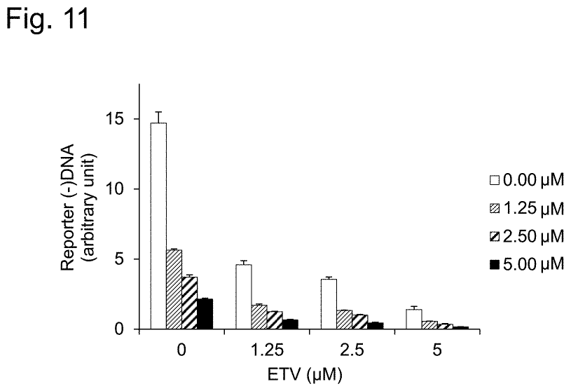

The results of synergetic effects are shown in FIG. 11. It turned out that in the cases where Entecavir and Hypothemycin are added, at any concentration, the replication-inhibiting effect of HBV DNA is enhanced in a concentration dependent manner than the case where Entecavir or Hypothemycin was added alone. As the result, it was found that Hypothemycin and Entecavir synergistically enhance the replication-inhibiting effect of HBV DNA.

### Example 9: HBV DNA Replication-Inhibiting Effect of MAPKK Inhibitor on Nucleotide/Nucleoside Analog Drug-Resistant HBV

### (Purpose)

HBV DNA replication-inhibiting effect of MAPKK inhibitor on mutant HBV-Pol, which causes the emergence of nucleotide/nucleoside analog-resistant HBV, is studied.

### (Method)

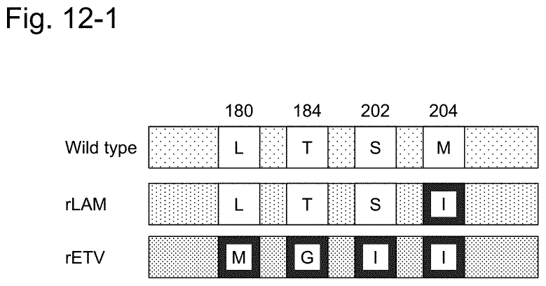

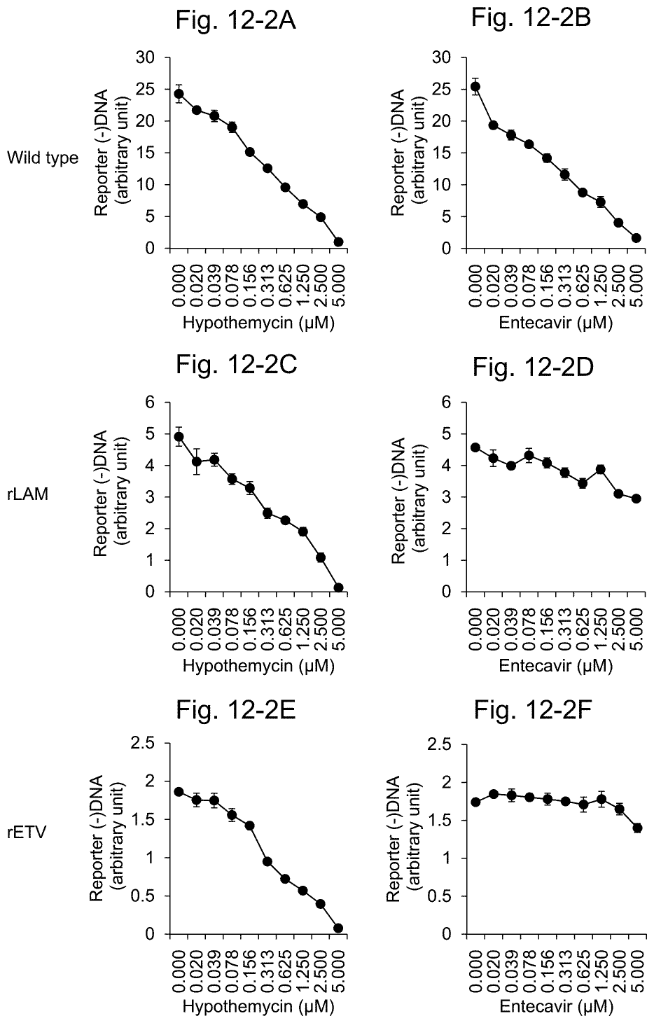

As described in the section of “background art”, long-term administration of conventional anti-HBV drugs made of a nucleotide/nucleoside analog such as Entecavir and Lamivudine causes the emergence of drug-resistant HBV. As a cause of the emergence of such a drug-resistant HBV, a mutation within the region of reverse transcription enzyme (RT) of HBV-Pol has been reported. For example, as shown in FIG. 12A, Lamivudine resistant HBV (LAMr) having an M204I mutation produced by substituting an amino acid, methionine (M) at position 204 positioned within the RT region with isoleucine (I); and Entecavir resistant HBV (ETVr) having a total of 4 mutations: L180M/T184G/S202I/M204I mutations, produced by substituting amino acids, leucine (L) at position 180 with methionine, T at position 184 with glycine (G), serine (S) at position 202 with I and M at position 204 with I positioned within the RT region, are known.

Then, mutant HBV-Pol expression vectors having M204I mutation, and L180M/T184G/S202I/M204I mutations respectively introduced therein were prepared in accordance with a conventional method and designated as HBV-P-LAMr, and HBV-P-ETVr, respectively. As the MAPKK inhibitor, Hypothemycin was used. As a negative control against a drug-resistant HBV, an existing anti-HBV drug, Entecavir, was used.

HBV replication evaluation was carried out in the same manner as in the evaluation method for HBV replication activity described in Example 1. The evaluation vector for HBV replication activity, and expression vectors of a wild type HBV-P or mutant HBV-P (HBV-P-LAMr or HBV-P-ETVr), HBc and HBx were introduced in the same ratio as in Example 2; at the same time, Hypothemycin or Entecavir was introduced in amounts of 0.000 μM, 0.020 μM, 0.039 μM, 0.078 μM, 0.156 μM, 0.313 μM, 0.625 μM, 1.250 μM, 2.500 μM or 5.000 μM. Culture was carried out under a 5% CO₂ atmosphere at 37° C. Twenty-four hours after culture, DNA was extracted from each of the culture solutions in accordance with the method described in Example 1 and subjected to real time PCR to study an inhibitory effect on HBV replication activity.

### (Results)

The results of inhibitory effect on HBV replication activity are shown in FIG. 12B. Although Entecavir suppressed reverse transcription by a wild-type HBV-Pol in a concentration-dependent manner (b), the suppression effect thereof on the reverse transcription activity of Lamivudine-resistance mutant HBV-Pol was weak (d). The reverse transcription activity of Entecavir-resistance mutant HBV-Pol was not substantially suppressed (f). In contrast, Hypothemycin suppressed the reverse transcription activities by wild type (a) and two drug-resistance mutant HBV-Pols (HBV-Pol-LAMr (c) and HBV-Pol-ETVr (e)) all in a concentration-dependent manner. From the results, it is demonstrated that HBV replication inhibitory action of a MAPKK inhibitor such as Hypothemycin differs in the mode of action from the anti-HBV activity of a nucleotide/nucleoside analog, more specifically, HBV replication inhibitory action based on suppression of the reverse transcription activity of HBV-Pol.

In chronic hepatitis B treatment, a problem currently concerned is “recurrence”, more specifically, reactivation of a virus. In particular, recurrence caused by a long-term administration treatment with a nucleotide/nucleoside analog formulation as typified by Entecavir, causes emergence of a drug-resistant HBV that makes the following treatment remarkably difficult. A substance inhibiting phosphorylation of residues T and Y in a TxY motif present within Terminal protein region of HBV-Pol, for example, a MAPKK inhibitor, is different in the mode of action from a nucleotide/nucleoside analog formulation. Thus, the substance such as an MAPKK inhibitor is applicable to recurrence causing emergence of drug-resistant HBV currently known and the effect thereof can be expected.

Accordingly, it was strongly suggested that the therapeutic agent for hepatitis B containing the MAPKK inhibitor of the present invention or the like as an active ingredient is extremely useful as a novel anti-HBV drug.

### Example 10: Experiment for Evaluating HBV Replication Using Deleted Forms of Evaluation Vectors for HBV Replication Activity

### (Purpose)

An experiment for evaluating HBV replication was carried out by using various deleted forms of evaluation vectors for HBV replication activity (ΔpBB-intron) to specify a sequence playing an important role in reverse transcription of pgRNA among the sequences derived from the HBV genome contained in pBB-intron.

### (Method)

The following 4 types of ΔpBB-introns were prepared.
(1) pBB-intron (Δε 1): first ε-deleted pBB-intron prepared by deleting a first ε sequence from the pBB-intron prepared in Example 1
(2) pBB-intron (ΔDR2): DR2-deleted pBB-intron prepared by deleting DR2 sequence from the pBB-intron prepared in Example 1
(3) pBB-intron (ΔDR1(2)): DR1(2)-deleted pBB-intron prepared by deleting second DR1 from the pBB-intron prepared in Example 1
(4) pBB-intron (Δε 2): second ε-deleted pBB-intron prepared by deleting a second c sequence from the pBB-intron prepared in Example 1

HeLa cells were transfected with pBB-intron (referred to as wild type pBB-intron in contrast to ΔpBB-intron) and one of the aforementioned 4 types of ΔpBB-introns together with HBV protein CP (C: HBc, P: HBV-Pol) or CPX (C: HBc, P: HBV-Pol, X: HBx). Twenty-four hours after culture, DNA was extracted and subjected to real time PCR. The amount of reporter HBV DNA reverse transcribed was quantified. The numerical value thereof is expressed as a ratio relative to the amount of genomic DNA (G3PDH exon 8) of a host (HeLa cells) quantified by real time PCR.

### (Results)

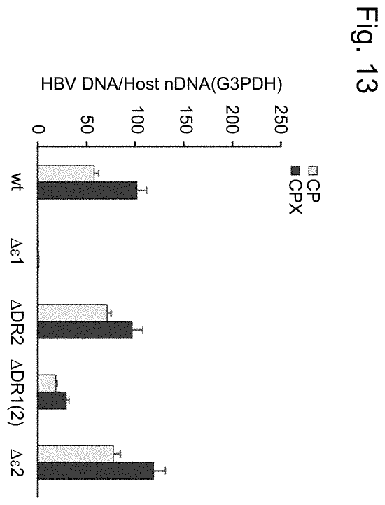

The results are shown in FIG. 13. In pBB-intron (Δε 1) prepared by deleting a first c sequence positioned on the 5′ terminal side, reporter HBV DNA almost completely disappeared. It was shown that the first ε sequence is an essential element for reverse transcription of pgRNA. In pBB-intron (ΔDR1(2)) prepared by deleting a second DR1 sequence positioned on the 3′ terminal side, the amount of reporter HBV DNA produced decreased. From this, it was shown that the second DR1 sequence is not essential for reverse transcription of pgRNA; however, it has a possibility to play an important role. In contrast, in the cases of pBB-intron prepared by deleting DR2 sequence (ΔDR2) and pBB-intron prepared by deleting the second ε sequence (Δε 2), the amounts of reporter HBV DNA produced did not significantly differ from that of wild type pBB-intron (wt). From the result, it was found that DR2 sequence and the second ε sequence are not particularly important at least in reverse transcription of pgRNA.

All publications, patents and patent applications cited in the specification are incorporated herein in their entireties by reference.

1. A method for evaluating replication activity in hepatitis B virus, comprising:
an introduction step of introducing into a host cell;
a vector comprising a promoter capable of inducing gene expression in a host cell and a nucleic acid comprising an epsilon sequence derived from hepatitis B virus, wherein the nucleic acid comprises any reporter sequence comprising an intron on the 3′ terminal side of the epsilon sequence, and is placed downstream of the promotor in a state that allows for the expression of the vector,
a hepatitis B virus polymerase expression vector in which a P gene from hepatitis B virus is placed in a state that allows for the expression downstream of a promoter capable of inducing gene expression in the host cell, and
a hepatitis B virus core protein expression vector in which a C gene from hepatitis B virus is placed in a state that allows for the expression downstream of a promoter capable of inducing gene expression in the host cell;
a culturing step of culturing the host cell after the introduction step;
an extraction step of extracting DNA from the host cell after the culturing step; and
a detection step of detecting a gene product of the reporter sequence in the vector using a primer pair consisting of a forward primer and a reverse primer designed to be capable of detecting the reporter sequence, in reporter minus strand DNA, wherein the gene product can be comprised in the DNA obtained in the extraction step and is the reporter sequence from which the intron has been removed from pre-mRNA of the reporter minus strand DNA.

2. The method according to claim 1, the introduction step further comprising introducing into the host cell a hepatitis B virus X protein expression vector in which an X gene from hepatitis B virus is placed in a state that allows for the expression downstream of the promoter capable of inducing gene expression in the host cell.

3. The method according to claim 1, wherein either of the forward primer or the reverse primer is designed to extend across the exon junction in the reporter sequence, so that only the reporter sequence in the reporter minus DNA is amplified.

4. The method according to claim 1, wherein the reporter minus strand corresponds to an HBV (−) strand DNA.

5. The method according to claim 1, wherein the nucleic acid comprising an epsilon sequence derived from hepatitis B virus serves as a template for the synthesis of a reporter pgRNA by RNA pol II when expressed in a host cell, and the intron in the reporter sequence is removed (spliced out) by pre-mRNA splicing upon synthesis of the reporter pgRNA.

6. The method according to claim 1, wherein the reporter minus strand DNA is generated only when the replication of HBV progresses and the amount of synthesized reporter minus strand DNA reflects the amount of synthesized minus strand DNA by the reverse transcription activity of HBV-Pol using pgRNA as template.
